(19) United States
(12) Patent Application Publication
(10) Pub. No.: US 2026/0258477 A1
(43) Pub. Date: Sep. 3, 2026

(54) DUAL QUENCHING ASSAY FOR MULTIPLEX DETECTION OF TARGET NUCLEIC ACIDS

(71) Applicant: Grifols Diagnostic Solutions Inc., Emeryville, CA (US)

(72) Inventors: Uffe Vest SCHNEIDER, Valby (DK); Søren Morgenthaler Echwald, Humlebæk (DK); Nikolaj Dam Mikkelsen, Brønshøj (DK)

(21) Appl. No.: 19/670,386

(22) Filed: May 7, 2026

Related U.S. Application Data
(63) Continuation of application No. 18/744,345, filed on Jun. 14, 2024.
Application No. 18/744,345 is a continuation of application No. 17/183,824, filed on Feb. 24, 2021, now abandoned.
Application No. 17/183,824 is a continuation of application No. 15/535,906, filed on Dec. 22, 2015, now abandoned.

(30) Foreign Application Priority Data
Dec. 22, 2014 (DK) PA201470813

(51) Int. Cl. C12Q 1/6818

(52) U.S. Cl. CPC C12Q 1/6818

(57) ABSTRACT

The present invention relates to a method for detecting at least one target nucleic acid sequence from a nucleic acid mixture by a double quenched assay. The double quenched assay of the method exploits a novel approach for melting temperature mediated identification of multiple target nucleic acid sequences. The invention further relates to a kit of parts.

### CROSS REFERENCE TO RELATED APPLICATIONS

This application is a continuation of U.S. application Ser. No. 17/183,824, filed Feb. 24, 2021, pending, which is a continuation of U.S. application Ser. No. 15/535,906, filed Jun. 14, 2017, abandoned, which is a National Stage of International Application No. PCT/DK2015/050412, filed Dec. 22, 2015, which claims the benefit of DK Application No. PA201470813, filed Dec. 22, 2014, each of which is incorporated herein in its entirety.

### REFERENCE TO SEQUENCE LISTING, TABLE OR COMPUTER PROGRAM

The instant application contains a Sequence Listing which has been filed electronically in XML format and is hereby incorporated by reference in its entirety. The XML copy, created on Jun. 11, 2024, is named 097091-0014-8001US02.XML and is 4900 bytes in size.

### FIELD OF INVENTION

The present invention relates to a qPCR method for indirect detection of multiple target DNA sequences based on melting temperature determination. The present invention further relates to a kit of parts.

### BACKGROUND OF INVENTION

Detection of specific sequences in a DNA sample by PCR has become a standard process. The technique is used for a range of different purposes from gene deletion analysis to pathogen identification and template quantitation. Typically fluorophores of different colors are used to detect different targets. However, due to the limited number of fluorophores which can easily be distinguished from one another, this gives a limitation in the number of specific targets that can be detected.

Furthermore, the step of DNA hybridization during the PCR reaction is affected by ionic strength, base composition, length of fragment to which the nucleic acid has been reduced, the degree of mismatching, and the presence of denaturing agents. DNA hybridization-based technologies are very useful tools in nucleic acid sequence determination and in clinical diagnosis, genetic research, and forensic laboratory analysis. A disadvantage, however, is that most of the conventional methods depending on hybridization are likely to produce false positive results due to non-specific hybridization between probes and non-target nucleic acid sequences. Therefore, there remain problems to be solved for improving their reliability.

Seegene, Seoul, Korea has developed a technology which can also accommodate multiplexing by melting curve analysis. As described in WO2013/115442A1, Seegene's TOCE technology is based on a probe which releases a primer fragment upon hydrolysis. This fragment is then required to act as a primer on a second, artificial target, where a double stranded target is generated, having a specific melting profile which can be linked to that particular probe. While this system can also accommodate high multiplexing by melting, it is inherently more complex than the present invention, by requiring the released fragment to initiate and complete a second extension on an artificial target. The fragment generated in the present invention will directly provide a labelled, melting fragment.

Pathofinder BV, Maastricht, Holland has developed a technology, which can also accommodate multiplexing by melting curve analysis. As described in United States Patent Application 20100297630 A1, this system is based on providing 2 target specific probes which, when hybridized adjacently on the target, can be conjoined by a ligase, which produces a melt-able fragment with a melting profile which is specific for the specific target probes. However, as indicated in United States Patent Application 20100297630 A1, this system is not a truly homogeneous assay but requires the sample tubes to be opened after the first PCR reaction to add e.g. a ligase solution. This extra step is not required in the method of the present invention and is highly undesired due to the risk of contamination by PCR product and also involves an additional handling step.

Roche Diagnostics has developed a method and assay for sepsis detection which also accommodates multiplexing by melting curve analysis. This system relies on FRET probes, where 2 probes having interacting labels are designed to bind adjacently to a single target, where the fluorophore of the first probe interacts with the second probe to generate a signal. The 2 probes can be designed such that the probes can generate a specific melting curve when subjected to a temperature gradient. A disadvantage by this system is that the melting profile must be designed within the specific target sequence, where the method of the present invention provides melting tags which, once optimized, can be attached to any target specific probes. In addition, since FRET probes require 2 labels, such a system can accommodate a lower level of multiplexing on a PCR system with e.g. 5 fluorophore detection channels compared to the present invention which carries one fluorophore per probe.

Other methods for multiplex DNA target detection are dependent on solid surface conjugation e.g. by chip based probes. Even though these chip based methods may be able to distinguish numerous target nucleic acid sequences, the chip-assembly process is cumbersome and often involve complicated, expensive and delicate equipment.

Therefore, there remains a need for convenient, reliable, and reproducible detection of multiple target nucleic acid sequences. Furthermore, a novel target detection method not limited by the number of fluorescent labels is needed. In addition there is a need in to reduce the complexity and number of steps in multiplex nucleic acid sequence analysis and thus facilitate more cost-effective and simple clinical diagnostic methods, genetic research protocols, and forensic laboratory analyses.

### SUMMARY OF INVENTION

The present invention provides a simple, convenient, reliable, and reproducible method of detecting multiple target DNA sequences. The present invention uses melting curve determinations for the detection of several target sequences per fluorophore. By this method a multitude of targets can be detected with a single fluorophore and/or a large number of targets can be detected by both employing different melting temperatures and different fluorophores. The present inventors have developed a dual quenched assay in combination with melting curve determinations for multiplex detection of target DNA sequences using different probing and tagging oligonucleotides (PTOs) each comprising a different melting temperature dependent region (MTDR) together with a single capturing and quenching oligonucleotide (CQO). Using several CQOs and PTOs of the present invention further increase the number of target sequence which can be detected in a single assay. A concept of the present invention, termed the MeltPlex system, is illustrated in FIGS. 1A-1C.

The present invention prevents false positive detection of target nucleic acid sequences by a combination of: 1) sequence specific hybridization to a target nucleic acid sequence and 2) sequence specific enzymatic release of an activated Tag Duplex fragment required for target signal detection (FIGS. 1A-1C). This indirect measurement of target nucleic acid sequences through the presence of an activated Tag Duplex fragment ensures excellent accuracy and reduces if not completely overcomes the issues with false positive results.

One advantage of the MeltPlex system is that one CQO may detect multiple target sequences identified by several unique PTOs. For each target nucleic acid sequence to be detected, a PTO with a MTDR sequence (melting temperature dependent region) which is unique within PTO's with same fluorophore, is designed. Consequently the number of target nucleic acid sequences which can be detected using only a single fluorescent label is increased. Different PTOs with similar fluorophores that are compatible with the same CQO may be referred to as a PTO group since they contain similar and/or identical fluorescent labels. Each PTO group may be detected by a single CQO, which forms an activated Tag Duplex, wherein the activated Tag Duplex fragments are distinguished based on differences in melting temperature as a consequence of the unique MTDR on each PTO in the group. Several PTO groups may also be detected by a single CQO, optionally by including more than on quencher in the CQO to allow quenching of a broad range of fluorophores by the same CQO. Hence the present invention is able to increase the number of target nucleic acid sequences which can be detected in an assay without requiring different types of fluorescent labels for each detected nucleic acid target nucleic acid sequence. The present invention may apply several fluorescent labels for different PTO groups, which further increases the number of target nucleic acid sequences which can be detected in an assay using standard laboratory equipment.

In addition the CQOs of the present invention are independent of the target sequences. Thus CQOs, which have proven to yield reliable results in some assays can be re-used in other assays. Such re-use of probes may save significant resources when designing new assays.

Contamination is a major issue in PCR based technologies. One option to limit the risk of contamination is to use a technology which does not require re-opening of the reaction vial after the assay has started. The present invention does not require re-opening of the reaction vials after assay start and is consequently less prone to contaminations.

A major aspect of the present invention relates to a method for detecting a target nucleic acid sequence, said method comprises the steps of:
(a) hybridizing the target nucleic acid sequence with a PTO (Probing and Tagging Oligonucleotide); the PTO comprising (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher; wherein the targeting portion of the PTO can hybridize with the target nucleic acid sequence and the MTDR of the PTO is not hybridized with the target nucleic acid sequence;
(b) hybridizing said PTO with a CQO (Capturing and Quenching Oligonucleotide); wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex;
(c) contacting the Tag Duplex with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore, wherein the PTO fragment is hybridized with the capturing portion of the CQO;
(d) melting and/or hybridizing said activated Tag Duplex fragment to obtain a signal from the at least one fluorophore, and;
(e) detecting the activated Tag Duplex fragment by measuring the signal from the at least one fluorophore; wherein the signal is indicative of the presence of the target nucleic acid sequence.

Steps a) and b) of the method may occur in any order, i.e. the Tag duplex may be formed prior to the binding of the PTO/Tag duplex to the target nucleic acid.

In another embodiment, steps (c) and (b) are switched so the method comprises the steps of (a) hybridizing the PTO and target, (c) contacting the PTO and target with enzyme having nuclease activity thus releasing the activated PTO and (b) hybridizing the activated PTO with a CQO thus forming an activated Tag Duplex and then steps (d) and (e) follow as disclosed above.

The steps of the method may be repeated.

The presence of activated PTO or activated Tag Duplex is registered.

The temperature at which the Tag Duplex melts (step d) is registered.

The assay of the present invention has a multitude of applications. A non-exhaustive list of applications includes:
Human and/or veterinary diagnostics
Food and/or feed quality and safety
Environmental surveillance
Scientific research

The assay of the present invention may be sold as a kit of parts. Thus an aspect of the present invention relates to a kit of parts for detection of a target nucleic acid sequence as described herein, the kit comprising:
i. optionally at least one PTO described herein, and
ii. at least one CQO described herein, and
iii. instructions on how to detect a target nucleic acid sequence.

An embodiment of the present invention is thus the detection of one or more target nucleic acid sequences with a single CQO.

### DESCRIPTION OF DRAWINGS

FIGS. 1A-1C: A non-limiting illustration of one embodiment of the present invention.

FIG. 2: Melting curve analysis of NTC (no template control) PCR reaction products: DEC486P does not generate a melting curve in the negative control as desired, whereas all other designs show increasing levels of melting curve signal in NTC reactions. (Results were performed in triplicates—singleplex shown). ◯: DEC486P, Δ: DEC487P, x: DEC488P, □: DEC489P, ⋄: DEC490P.

FIG. 3: Melting curve analysis of PCR reaction products with and without template for tagging probe DEC486P. NTC (X) does not generate a melting curve in the negative control, whereas there is a clearly distinguishable signal from the reaction with template (⋄). Also, no melting curve is seen in reaction without quenching probe (O). (Results were performed in triplicates—singleplex shown).

FIG. 4: Melting curve analysis of PCR reaction products with and without template for tagging probe DEC487P. NTC (X) generates a small melting curve in the negative control, whereas there is a clearly distinguishable signal from the reaction with template (⋄). No melting curve is seen in reaction without quenching probe (O). (Results were performed in triplicates—singleplex shown).

FIG. 5: Melting curve analysis of PCR reaction products with and without template for tagging probe DEC488P. NTC (X) generates a modest melting curve in the negative control, whereas there is a clearly distinguishable signal from the reaction with template (⋄). No melting curve is seen in reaction without quenching probe (O). (Results were performed in triplicates—singleplex shown).

FIG. 6: Melting curve analysis of PCR reaction products with and without template for tagging probe DEC489P. NTC (X) generates a clear melting curve in the negative control, whereas there is a distinguishable signal from the reaction with template (⋄). No melting curve is seen in reaction without quenching probe (O). (Results were performed in triplicates—singleplex shown).

FIG. 7: Melting curve analysis of PCR reaction products with and without template for tagging probe DEC490P. NTC (X) generates a significant melting curve in the negative control, which is not clearly distinguishable from the reaction with template (⋄). No melting curve is seen in reaction without quenching probe (O). (Results were performed in triplicates—singleplex shown).

FIG. 8A: Q-PCR amplification curves for tagging probes DEC486P−DEC490P on positive samples. ◯: DEC486P, Δ: DEC487P, x: DEC488P, □: DEC489P, ⋄: DEC490P, full line: DEC464. Although signal intensity is lower for tagging probes compared to the TaqMan-type probe, all probes clearly generate positive signal readouts with CT values of between 16 and 20.

FIG. 8B: A non-limiting illustration of one embodiment of the present invention.

FIG. 9: Melting curve analysis of target positive and NTC (no template control) PCR reaction products: DEC500P generates a melting curve in the positive sample but not in the negative control as desired (results were performed in triplicates—singleplex shown). □: DEC500P, -: DEC500P NTC (non template control)

FIG. 10: Amplification curve of target positive and NTC (no template control) PCR reaction products: DEC500P generates an amplification curve in the positive sample but not in the negative control as desired (results were performed in triplicates—singleplex shown). □: DEC500P, -: DEC500P NTC (non template control)

FIG. 11: Melting curve analysis of target positive and NTC (no template control) PCR reaction products: DEC502P generates a melting curve in the positive sample but not in the negative control as desired (results were performed in triplicates—singleplex shown). □: DEC500P, ⋄: DEC502P, -

FIG. 12: Amplification curve of target positive and NTC (no template control) PCR reaction products: DEC502P generates an amplification curve in the positive sample but not in the negative control as desired (results were performed in triplicates—singleplex shown). □: DEC500P, ⋄: DEC502P, -: DEC502P NTC (non template control)

FIG. 13: Melting curve analysis of target positive and NTC (no template control) PCR reaction products: DEC503P generates a melting curve in the positive sample but not in the negative control as desired (results were performed in triplicates—singleplex shown). x: DEC503P, -: DEC503P NTC (non template control)

FIG. 14: Amplification curve of target positive and NTC (no template control) PCR reaction products: DEC503P generates an amplification curve in the positive sample but not in the negative control as desired (results were performed in triplicates—singleplex shown). x: DEC503P, -: DEC503P NTC (non template control)

FIG. 15: Melting curve analysis of target positive and NTC (no template control) PCR reaction using DEC500P and DEC502P generates 2 individually distinguishable melting curve in the positive sample but not in the negative control as desired (results were performed in triplicates—singleplex shown). ◯: DEC500P+DEC 502P, -: DEC500P+DEC 502P NTC (non template control).

FIG. 16: Amplification curve of target positive and NTC (no template control) PCR reaction products using DEC500P and DEC502P generates a amplification curve in the positive sample but not in the negative control as desired (results were performed in triplicates—singleplex shown). ◯: DEC500P+DEC 502P, -: DEC500P+DEC 502P NTC (non template control).

FIG. 17: Melting curve analysis of target positive and NTC (no template control) PCR reaction using DEC500P and DEC503P generates 2 individually distinguishable melting curve in the positive sample but not in the negative control as desired (results were performed in triplicates—singleplex shown). Δ: DEC500P+DEC 503P, -: DEC500P+DEC 503P NTC (non template control)

FIG. 18: Amplification curve of target positive and NTC (no template control) PCR reaction products using DEC500P and DEC503P generates a amplification curve in the positive sample but not in the negative control as desired (results were performed in triplicates—singleplex shown). Δ: DEC500P+DEC 503P, -: DEC500P+DEC 503P NTC (non template control)

FIG. 19: Melting curve analysis of target positive and NTC (no template control) PCR reaction products: RMD7P generates a melting curve in the positive sample but not in the negative control as desired (results were performed in triplicates—singleplex shown). O: RMD7P, -: RMD7P NTC (non template control).

FIG. 20: Amplification curve of target positive and NTC (no template control) PCR reaction products: RMD7P generates an amplification curve in the positive sample but not in the negative control as desired (results were performed in triplicates—singleplex shown). O: RMD7P, -: RMD7P NTC (non template control).

FIG. 21: Melting curve analysis of target positive and NTC (no template control) PCR reaction products: RMD8P generates a melting curve in the positive sample but not in the negative control as desired (results were performed in triplicates—singleplex shown). X: RMD8P, -RMD8P NTC (non template control).

FIG. 22: Amplification curve of target positive and NTC (no template control) PCR reaction products: RMD8P generates an amplification curve in the positive sample but not in the negative control as desired (results were performed in triplicates—singleplex shown). X: RMD8P, -: RMD8P NTC (non template control).

FIG. 23. Averaged Cq values of the RMD assay, plotted as a function of polymerase concentration. X-axis: arbitrary units.

FIG. 24. Averaged Cq values of the mValidPrime assay, plotted as a function of polymerase concentration. X-axis: arbitrary unit, where 1 is the concentration recommended by the manufacturer.

FIG. 25. Amplitude of the RMD amplification curves in step 2 (95° C.) as a function of polymerase concentration. X-axis: arbitrary unit, where 1 is the concentration recommended by the manufacturer.

FIG. 26. Amplitude of the mValidPrime amplification curves in step 2 (95° C.) as a function of polymerase concentration. X-axis: arbitrary unit, where 1 is the concentration recommended by the manufacturer.

FIG. 27. Melting temperatures of the RMD probe/quencher duplexes as a function of polymerase concentration. X-axis: arbitrary unit, where 1 is the concentration recommended by the manufacturer.

FIGS. 28A-28B: (A) Amplification curve of target positive and NTC (no template control) PCR reaction products: DEC486P generates amplification curves with Cq values in the positive samples corresponding to the target concentration but not in the negative control as desired (results were performed in duplicates—singleplex shown). Δ: 5× dilution, x: 25× dilution, □: 125× dilution, ⋄: 625× dilution, O: 3125× dilution, -: 15625× dilution, NTC: (non template control). (B) Melting curve analysis of target positive and NTC (no template control) PCR reaction products: DEC486P generates melting curves amplitudes in the positive samples corresponding to the target concentration but not in the negative control as desired (results were performed in duplicates—singleplex shown). Δ: 5× dilution, x: 25× dilution, □: 125× dilution, ⋄: 625× dilution, O: 3125× dilution, -: 15625× dilution, NTC: (non template control).

### DETAILED DESCRIPTION OF THE INVENTION

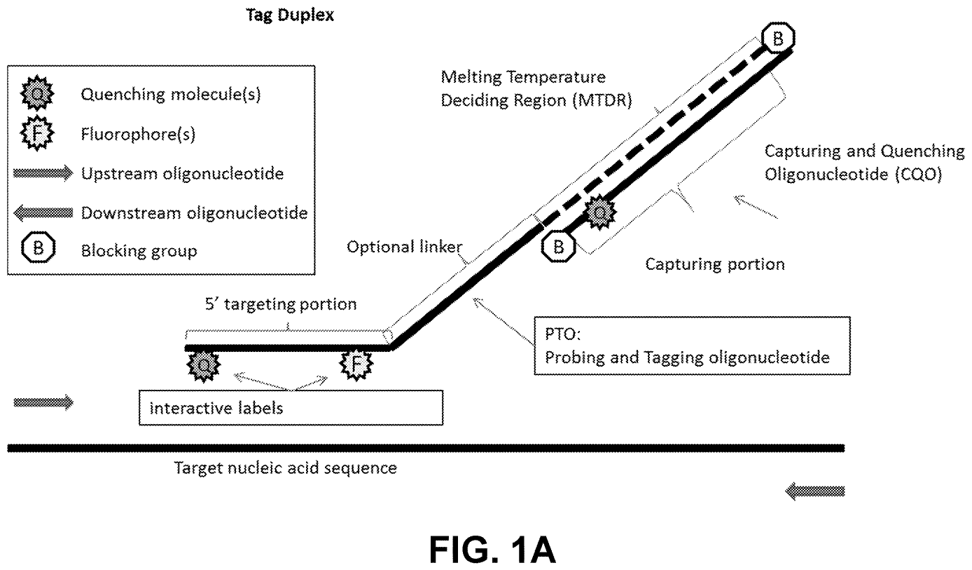

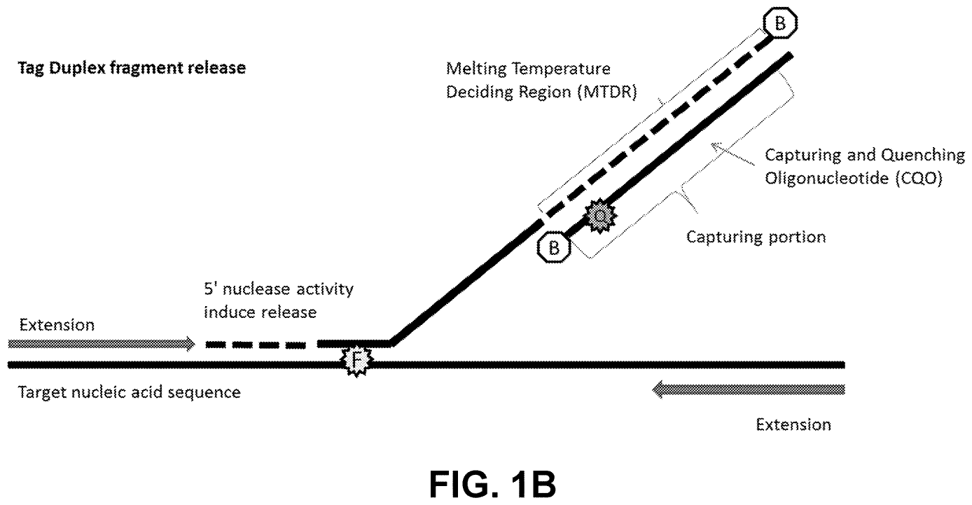

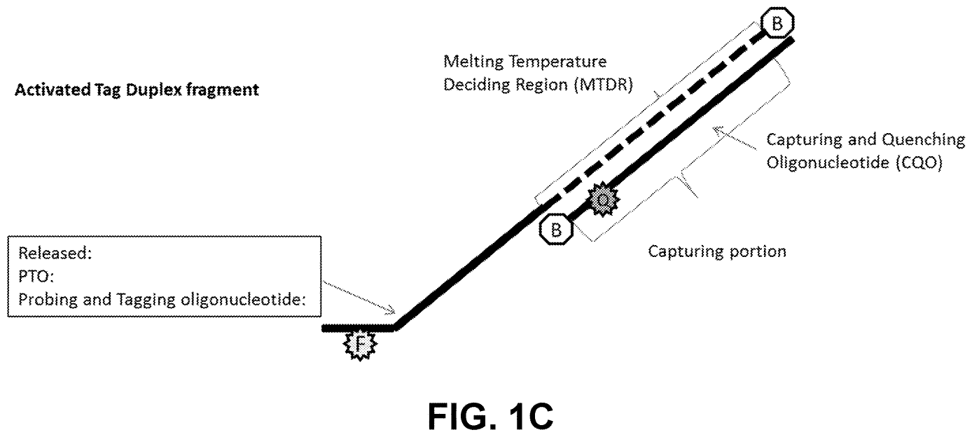

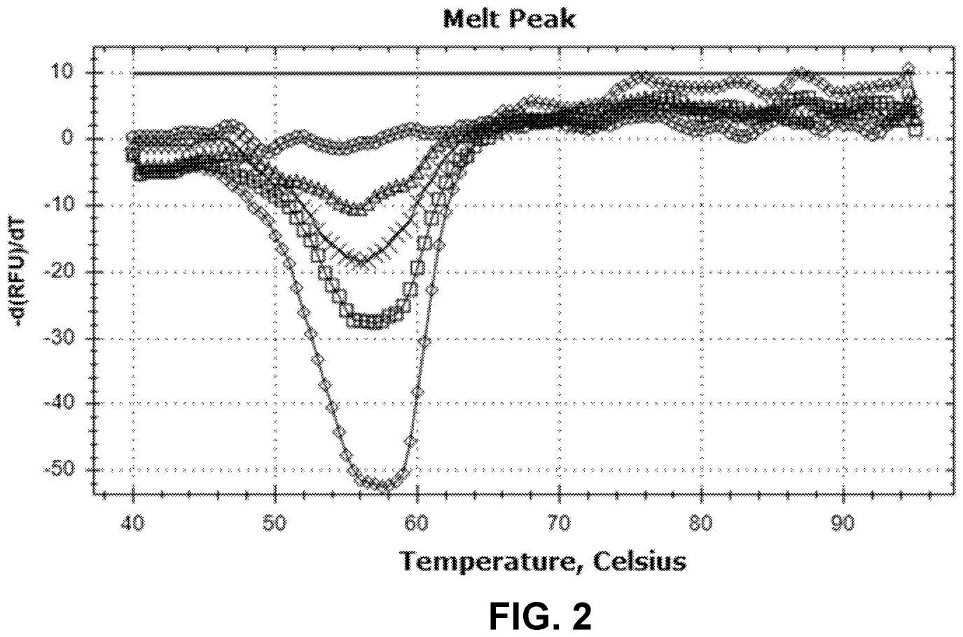

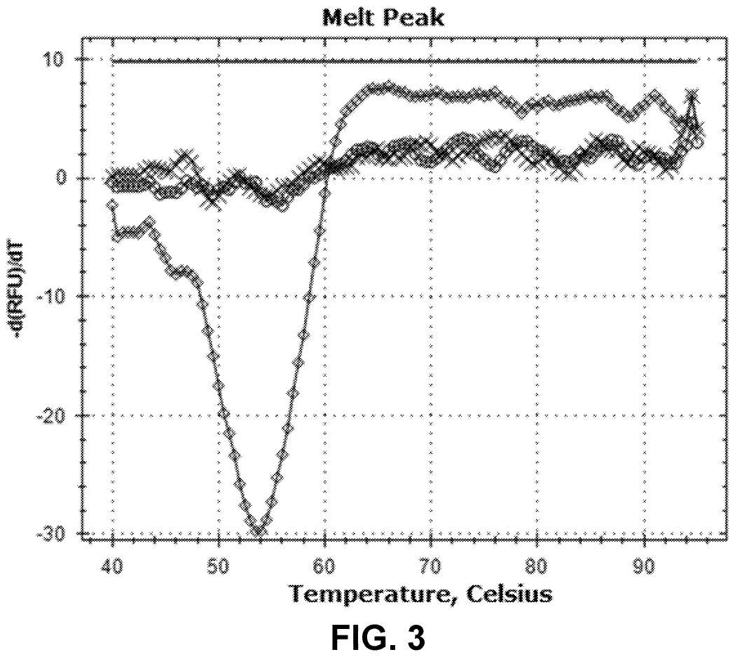

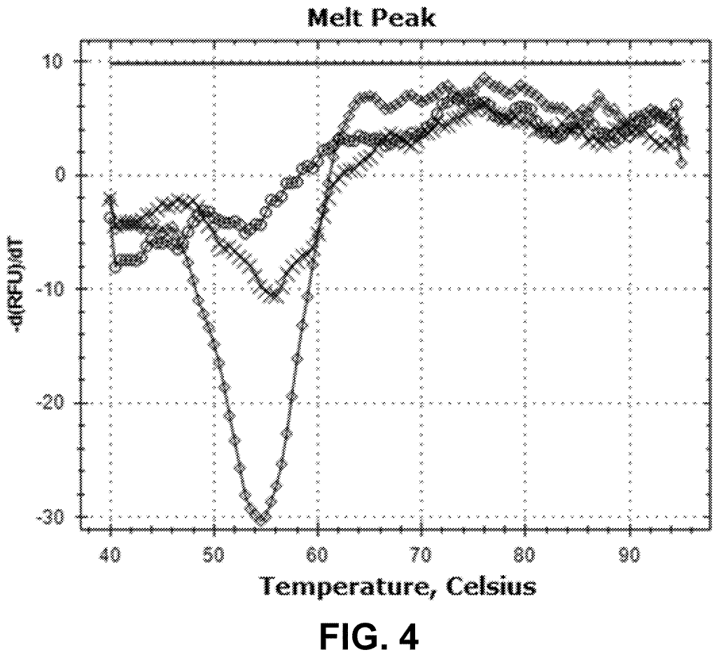

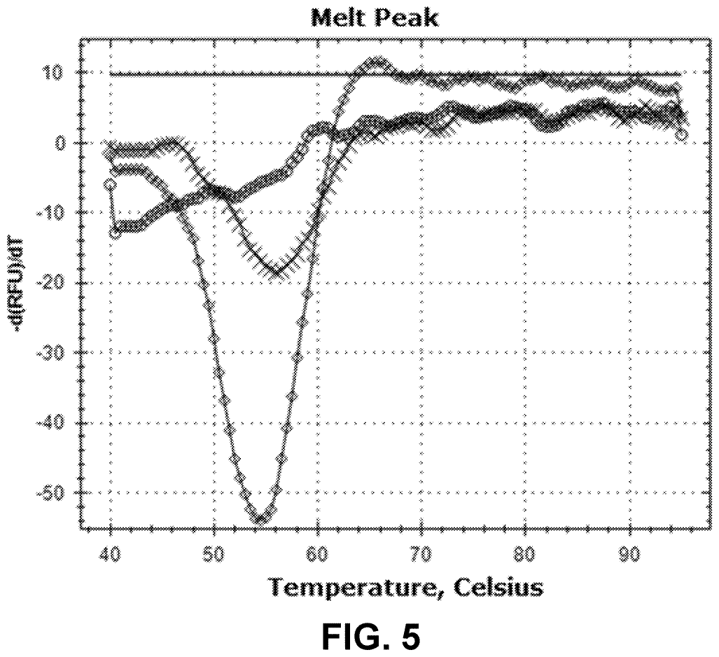

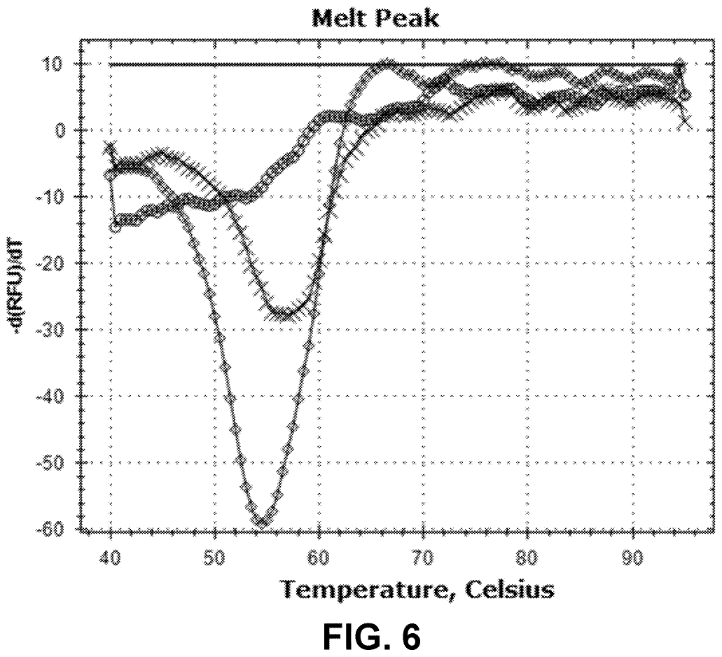

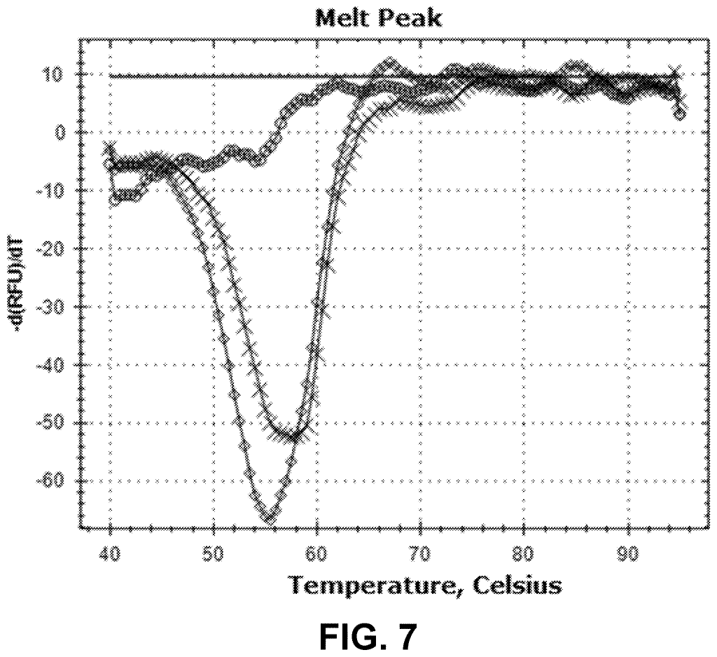

The major challenges of multiplex PCR is easy detection of multiple target DNA sequences using a simple, convenient, and reliable method. The present inventors have developed a dual quenched assay in combination with melting curve determination for detection of several target DNA sequences per label, i.e. per fluorophore. A non-limiting concept of the present invention is illustrated in FIGS. 1A-1C.

### Definitions

The term “double quenched assay” as used herein refers to the use of at least two quenchers for at least one fluorophore. In an embodiment one quencher is situated on the CQO and another quencher is situated on the PTO. In an embodiment the fluorophore is situated on the PTO.

The term “interactive labels” or “set of interactive labels” as used herein refers to at least one fluorophore and at least one quencher which can interact when they are located adjacently. When the interactive labels are located adjacently the quencher can quench the fluorophore signal. The interaction may be mediated by fluorescence resonance energy transfer (FRET).

The term “located adjacently” as used herein refers to the physical distance between two objects. If a fluorophore and a quencher are located adjacently, the quencher is able to partly or fully quench the fluorophore signal. FRET quenching may typically occur over distances up to about 100 Å. Located adjacently as used herein refers to distances below and/or around 100 Å.

The term “probing and tagging oligonucleotide” or “PTO” as used herein refers to an oligonucleotide comprising at least one set of interactive labels. A PTO of the present invention is configured to hybridize to a target nucleic acid sequence. A PTO comprises a targeting portion, a “Melting Temperature Deciding Region” or “MTDR” (see definition below), and optionally a linker between the targeting portion and the MTDR.

The term “PTO group” as used herein refers to a number of PTOs with the same set of interactive labels, wherein each PTO in the group has a unique targeting sequence and MTDR region. Each PTO in a group may be configured to detect different target nucleic acid sequences and the unique MTDR facilitates distinction of each PTO in the group by means of melting temperature as described herein.

The term “Capturing and quenching oligonucleotide” or “CQO” as used herein refers to an oligonucleotide comprising at least one quencher and a capturing portion. The capturing portion of the CQO is configured to hybridize to a PTO of the present invention.

The term “Tag duplex” as used herein refers to a PTO and a CQO which are hybridized. The PTO may furthermore be hybridized to a target nucleic acid sequence.

The term “Tag Duplex fragment” or “activated Tag Duplex fragment” or activated Tag Duplex as used herein refers to a PTO fragment and a CQO which are hybridized, wherein the quencher of the PTO is not present. The quencher of the PTO has been released as a consequence of the enzyme having nuclease activity which induces cleavage of the Tag Duplex and release of the PTO quencher. “Activated PTO” refers to the PTO where the quencher has been removed. The presence of activated PTO may be measured using qPCR and/or real-time PCR. Dependent on the sequence of the steps of the methods disclosed herein the presence of either activated PTO or activated Tag Duplex is measured. In preferred embodiments only activated Tag Duplex can be detected by real time PCR. In the most preferred embodiments only the activated Tag Duplex can be detected by real time PCR due to complete quenching of the PTO fluorophore by the CQO quencher. In other embodiments the assay is calibrated after any signal detected by an activated PTO.

The term “fluorescent label” or “fluorophore” as used herein refers to a fluorescent chemical compound that can re-emit light upon light excitation. The fluorophore absorbs light energy of a specific wavelength and re-emits light at a longer wavelength. The absorbed wavelengths, energy transfer efficiency, and time before emission depend on both the fluorophore structure and its chemical environment, as the molecule in its excited state interacts with surrounding molecules. Wavelengths of maximum absorption (~excitation) and emission (for example, Absorption/Emission=485 nm/517 nm) are the typical terms used to refer to a given fluorophore, but the whole spectrum may be important to consider.

The term “quench” or “quenching” as used herein refers to any process which decreases the fluorescence intensity of a given substance such as a fluorophore. Quenching may be mediated by fluorescence resonance energy transfer (FRET). FRET is based on classical dipole-dipole interactions between the transition dipoles of the donor (e.g. fluorophore) and acceptor (e.g. quencher) and is dependent on the donor-acceptor distance. FRET can typically occur over distances up to 100 Å. FRET also depends on the donor-acceptor spectral overlap and the relative orientation of the donor and acceptor transition dipole moments. Quenching of a fluorophore can also occur as a result of the formation of a non-fluorescent complex between a fluorophore and another fluorophore or non-fluorescent molecule. This mechanism is known as ‘contact quenching,’ ‘static quenching,’ or ‘ground-state complex formation

The term “quencher” as used herein refers to a chemical compound which is able to quench a given substance such as a fluorophore.

In multiplex PCR more than one target nucleic acid sequence may be detected.

The term “Melting Temperature Deciding Region” or “MTDR” as used herein refers to a polynucleotide region located in the 5′ end of the PTO. The nature and/or the number of polynucleotides in the MTDR are decisive for the melting temperature of e.g. the activated Tag Duplex comprising a PTO fragment and a CQO. Likewise the MTDR is decisive for the hybridization temperature of e.g. the activated Tag Duplex comprising a PTO fragment and a CQO.

The term “melting temperature” or “Tₘ” as used herein refers to the temperature at which one half of a DNA duplex will dissociate to become single stranded and thus indicates the duplex stability. The main factors affecting Tₘ are salt concentration, DNA concentration, pH and the presence of denaturants (such as formamide or DMSO). Other effects such as sequence, length, and hybridization conditions can be important as well. The GC content of the sequence and the salt concentration gives a fair indication of the primer Tₘ. The melting temperatures referred to in the present invention are calculated using the nearest neighbor thermodynamic theory as described by Kibbe et al. 2007. The corresponding Tₘ calculator is available at the URL: http://basic.northwestern.edu/biotools/OligoCalc.html. The Tₘ values given in the present invention have been calculated on the basis of 800 nm CQO (“Primer”) and 50 nm (Na⁺). In melting temperature calculations of oligos comprising analogs of adenine, thymine, cytosine and/or guanine the analog is replaced by its corresponding nucleic acid. Fluorophore and quenchers on the oligos should not be considered when calculating the melting temperature. Determination of the melting temperature may be performed either by heating a DNA duplex or by cooling (hybridizing) two single stranded DNA strands which are substantially complementary.

The term “denaturation” as used herein is the dissociation by disrupting the hydrogen bonds between complementary bases of DNA to become single stranded. It may also refer to a cycling event of a PCR reaction and may e.g. comprise heating the reaction to 90-100° C. for 3-240 seconds.

The term “ready to use pellet” as used herein refers to a substantially water free composition comprising at least one PTO and/or at least one CQO of the present invention.

The term “background melting curve generation” as used herein refers to background signals during melting curve analysis. A background signal may occur if the signal of the at least one fluorophore on the PTO is not completely quenched by the at least one quencher of the PTO.

The term “TINA” as used herein, refers to a twisted intercalating nucleic acid and is a group of nucleic acid intercalating molecules as described in U.S. Pat. No. 9,102,937.

The term “locked nucleic acid” (LNA) as used herein refers to a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties of oligonucleotides by increasing the thermal stability of duplexes. LNAs and methods of synthesis thereof are known to the skilled person, and are described e.g. in European patents EP1015469 and EP1015469.

The term ‘reverse complementary’ as used herein designates a nucleic acid sequence which is capable of hybridizing to another nucleic acid sequence of which it is the reverse complement. For example, the reverse complement of a sequence 5′-N₁N₂N₃N₄ . . . Nₓ-3′ is 5′-Nₓ′ . . . N₄′N₃′N₂′N₁′-3′, where Nₓ′, N₄′, N₃′, N₂′, N₁′ indicate the nucleotides complementary to Nₓ, N₄, N₃, N₂, N₁, respectively.

### Target Detection

The present invention relates to a novel dual quenching assay which allows simultaneous detection of multiple target nucleic acids per fluorescent label. The presence of multiple target nucleic acid sequences in a sample results in the formation of multiple Tag Duplex fragments which can be distinguished based on differences in Tag Duplex fragment melting temperatures and/or hybridization temperature. The Tag Duplex comprises a PTO and a CQO which are hybridized. When each activated Tag Duplex fragment melts, a signal from a label of the PTO is obtained. A signal at a specific temperature is thus indicative of the presence of the target sequence that the PTO is specific for. When each activated Tag Duplex fragment hybridizes, a signal from a label of the PTO is quenched. A quenched signal at a specific temperature is thus indicative of the presence of the target sequence that the PTO is specific for. The general concept of the present invention is illustrated in FIGS. 1A-1C. The present invention prevents false positive signals by a combination of 1) sequence specific hybridization to the target nucleic acid sequence by means of the PTO targeting portion and 2) sequence specific enzymatic release of an activated Tag Duplex fragment required for target signal detection, by means of the nuclease activity of a polymerase extending an oligonucleotide upstream of the target sequence. This indirect measurement of the target nucleic acid sequence through the presence of an activated Tag Duplex fragment ensures excellent accuracy and overcomes issues with false positives. A single CQO may hybridize to many PTOs and/or many groups of PTOs. Thus the present invention relates to the detection of one or more, such a multiple, nucleic acids with the aid of a single CQO.

### Melting Temperature Mediated Identification of Multiple Target Nucleic Acid Sequences

Using one CQO for detection of several PTO's with unique MTDRs results in a simpler assay setup which can detect several target nucleic acid sequences per fluorophore (e.g. per PTO group). The number of PTOs in a PTO group which can be distinguished using one fluorophore is dependent of the sensitivity of the analytical equipment used for detecting the signal of the fluorescent tag upon melting the activated Tag Duplex fragment. In a simple setup provided here by way of example: one CQO may be used to identify at least three PTOs of a PTO group; using two PTO groups with two different fluorophores may thus facilitate detection of at least 6 PTOs in a single reaction. Each PTO indicates the presence of a target nucleic acid sequence. The number of targets to be identified may be further increased by using three or more PTO groups with different fluorescent tags. A single CQO may of course be able to hybridize with more than two, such as three, four or more PTOs. A PTO group may comprise 2, 3, 4, 5, 6, 7 or more PTOs and a single CQO may be used to detect each of these PTOs. Simultaneously other PTO groups (PTOs with different flourophores) may be detected with the same CQO as well. In this manner a single CQO may be used to detect multiple targets. In an embodiment a single CQO may thus detect 2, 4, 6, 8, 10, 15, 20, 30, 40, 50, or more PTOs and thus the assay can detect the corresponding number of target nucleic acids.

The setup of the dual quenching assay of the present invention yields a simple reliable assay for multiplex detection of target nucleic acids. The simple assay of the present invention has a multitude of applications. A non-exhaustive list of applications of the assay of the present invention may be:
The use of the method of the present invention for human and/or veterinary diagnostics. e.g. clinical identification and/or quantification of a target nucleic acid from at least one microorganism, such as a pathogen, or oncogene or microsatellite in a bodily sample from a subject.
The use of the method of the present invention for environmental surveillance. E.g. identification and/or quantification of a target nucleic acid from at least one (micro)organism in any sample, for example a water sample.
The use of the method of the present invention for food and/or feed quality and safety determination e.g. identification and/or quantification of a target nucleic acid from at least one organism in any sample such as a feed and/or food product, such as a beverage sample.
The use of the method of the present invention for scientific research.

In a main aspect the present invention relates to a method for detecting a target nucleic acid sequence, the method comprising the steps of:
Step (a) hybridizing a target nucleic acid sequence with a PTO (Probing and Tagging Oligonucleotide); the PTO comprising (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher;
Step (b) hybridizing said PTO with a CQO (Capturing and Quenching Oligonucleotide); wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex;
Step (c) contacting the Tag Duplex with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore;
Step (d) melting and/or hybridizing said activated Tag Duplex fragment to obtain a signal from the at least one fluorophore, and
Step (e) detecting the activated Tag Duplex fragment by measuring the signal from the at least one fluorophore; wherein the signal is indicative of the presence of the target nucleic acid sequence.

Steps (a), (b) and (c) of any of the herein embodiments of the method may form part of a PCR reaction. A set of oligonucleotide primers is added that will amplify the target sequence. This amplification will due to the presence of a polymerase with exonuclease activity result in the release of the thus activated PTO or the activated Tag Duplex. The term activated relates to the absence of the quencher on the PTO. Any presence of activated PTO or activated Tag Duplex may be registered/detected. If the PCR reaction is a qPCR reaction the amount of activated PTO or activated Tag Duplex may be quantified. The oligonucleotide primer pair comprises a first a primer complementary to said target nucleic acid and which primes the synthesis of a first extension product that is complementary to said target nucleic acid, and a second primer complementary to said first extension product and which primes the synthesis of a second extension product. The PTO may hybridize to the target nucleotide sequence and inter alia to the amplification product.

In an embodiment the presence of activated PTO and/or activated Tag Duplex is detected. In another embodiment the amount of activated PTO and/or activated Tag Duplex is quantified. The detection and/or quantification of the presence of activated PTO and/or activated tag Duplex may be an optional step to be included once the activated PTO and/or activated Tag Duplex is formed.

Steps (d) and (e) form part of a melting assay in which the presence and/or absence targets is determined based on the Tm of the activated Tag Duplex. The melting assay may be run in a PCR machine.

Steps (a) and (b) may occur in any order. Thus in an embodiment of the present invention the method described herein comprises the steps of:
Step (b) hybridizing a PTO (Probing and Tagging Oligonucleotide) with a CQO (Capturing and Quenching Oligonucleotide); the PTO comprising (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher; and wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex;
Step (a) hybridizing a target nucleic acid sequence with a PTO of a Tag Duplex;
Step (c) contacting the Tag Duplex with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore;
Step (d) melting and/or hybridizing said activated Tag Duplex fragment to obtain a signal from the at least one fluorophore, and
Step (e) detecting the activated Tag Duplex fragment by measuring the signal from the at least one fluorophore; wherein the signal is indicative of the presence of the target nucleic acid sequence.

In yet an embodiment steps (b) and (c) occur in reverse order. Thus in an embodiment of the present invention the method described herein comprises the steps of:
Step (a) hybridizing a target nucleic acid sequence with a PTO (Probing and Tagging Oligonucleotide); the PTO comprising (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher;
Step (c) contacting the hybridized PTO with an enzyme having nuclease activity; wherein the enzyme having nuclease activity induces cleavage of the PTO when the PTO is hybridized with the target nucleic acid sequence thereby releasing an activated PTO fragment comprising the MTDR and the at least one fluorophore;
Step (b) hybridizing said activated PTO with a CQO (Capturing and Quenching Oligonucleotide); wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form an activated Tag Duplex;
Step (d) melting and/or hybridizing said activated Tag Duplex fragment to obtain a signal from the at least one fluorophore, and
Step (e) detecting the activated Tag Duplex fragment by measuring the signal from the at least one fluorophore; wherein the signal is indicative of the presence of the target nucleic acid sequence.

In all the assays the steps may be repeated. Particularly steps (a) and (c) in the orders indicated above for various assays may be repeated one or more times. The number of repetitions may be as is customary for performing PCR reactions.

The temperature at which the Tag Duplex melts in step (d) of the method is registered. Thus in an embodiment of the invention step (d) comprises:
Step (d) melting and/or hybridizing said activated Tag Duplex fragment to obtain a signal from the at least one fluorophore, and registering the melting temperature of the activated Tag Duplex fragment.

In an embodiment the method described above further comprises repeating the steps (a)-(b), (a)-(c), (a)-(d) and/or (a)-(e) with denaturation between repeating cycles. It follows that in an embodiment starting with step (b), steps (b)-(a), (b)-(c), and/or (b) to (e) are repeated with denaturation between repeating cycles. In another embodiment the steps are performed in one reaction vessel or some of the steps (a)-(e) or (b)-(e) are performed in one or more separate reaction vessels. In an embodiment starting with steps (a), (c) and (b) the steps may be repeated by repeating steps (a)-(c), (a)-(b), (a)-(d) and/or (a)-(e) with denaturation between repeating cycles. In a further embodiment of all the methods, the steps (a), (b) and (c) occur simultaneously or more or less simultaneously and/or steps (d) and (e) occur simultaneously or more or less simultaneously.

A non-limiting example illustrating the detection of two target sequences may be: A mixture comprising:
1) a PTO #1 comprising a targeting portion #1, and a MTDR #1 configured to yield a melting temperature of 50° C., wherein the PTO #1 can hybridize to target nucleic acid sequence #1, and
2) a PTO #2 comprising a targeting portion #2, and a MTDR #2 configured to yield a melting temperature of 60° C., wherein the PTO #2 can hybridize to a target nucleic acid sequence #2, and
3) a CQO which is configured to hybridize to MDTR #1 and MDTR #2 as described herein above.

In presence of the target nucleic acid sequence #1 and sequence #2 the method of the present invention yields an activated Tag Duplex fragment #1 wherein the CQO is hybridized to the MTDR #1 of the PTO #1 and an activated Tag Duplex fragment #2 wherein the CQO is hybridized to the MTDR #2 of the PTO #2. When melting a mixture comprising the activated Tag Duplex fragment #1 and Tag Duplex fragment #2 over a range of temperatures a first signal is generated when the temperature reaches 50° C. and a second signal is generated when the temperature reaches 60° C. In this simplified example the first signal (at 50° C.) is indicative of the presence of the target nucleic acid sequence #1 and the second signal is indicative of the presence of the of target nucleic acid sequence #2. Thus with one fluorophore it is possible to detect at least two different targets. A person of skill can easily see how a multitude of targets may be determined by the method of the present invention.

The present invention may further include at least one Tag Duplex or DNA duplex comprising at least one set of interactive labels comprising at least one fluorophore and at least one quencher which may be used as control sample for example for calibrating the Tₘ of the analytical equipment. The Tₘ of an oligonucleotide may vary depending on e.g. the salt concentration, DNA concentration, pH and the presence of denaturants (such as formamide or DMSO). Inclusion of a control sample may be desirable if the samples to be analyzed contain varying i.e. salt concentrations. In an embodiment the method described herein further comprises analyzing a control sample and/or control Tag Duplex. In another embodiment the method described herein further comprises analyzing a control sample and/or control Tag Duplex to calibrate the Tₘ output of the analytical equipment. It follows that in the absence of target sequence the present methods will detect the presence of PTO and tag Duplexes that are not activated by the removal of the quencher on the PTO. As is known to the person of skill: when running assays as those disclosed herein a negative control (no target present) and a positive control (presence of target) may be included. The controls may be used to calibrate the assay. An example of a commercially available control for the presence of genomic DNA is ValidPrime.

As is known to the person skilled in the art, the melting temperature of a duplex is usually not dependent on the nature of the sequence, but rather on the relative amounts of the individual nucleotides. The skilled person also knows to avoid particular sequences which might result in the generation of secondary or tertiary structures which might impede the reaction. As illustrated in the examples, the present methods work with MTDR having different sequences.

The MTDR comprises a nucleotide sequence non-complementary to the target nucleic acid sequence. The term ‘non-complementary’ in this context will be understood by the skilled person as referring to a sequence which is essentially non-complementary, i.e. essentially unable to hybridize to the target nucleic acid sequence under normal PCR conditions and/or stringent conditions.

Similarly, the term ‘a nucleotide sequence substantially complementary to the target nucleic acid sequence’ refers to a nucleotide sequence which is able to hybridize to the target nucleic acid sequence in such a manner that extension by a polymerase is efficient or even feasible. As will be obvious to the skilled person, there may be some mismatches, provided that they do not prevent hybridization of the nucleotide sequence to the target nucleic acid sequence to such an extent that extension by a polymerase is not possible.

### PTO

For each target nucleic acid sequence to be identified a PTO configured for hybridizing to each target nucleic acid sequence is obtained. Each PTO has a MTDR which is unique among PTO sharing same or similar fluorophore. The MTDR of the PTO is decisive for the activated Tag Duplex fragment melting temperature. Each Tag Duplex fragment comprises a PTO fragment with a unique MTDR and the at least one fluorophore, wherein the MTDR of the PTO fragment is hybridized with the capturing portion of the CQO. By unique MTDR is meant an MTDR which is unique in the melting temperature it confers to the Tag Duplex and/or Tag Duplex fragment. The unique MTDR is thus unique within a group of PTOs. A unique MTDR may thus be used in several PTOs each with different labels/fluorophores.

In an embodiment the targeting portion is located in the 5′ end of the PTO. In another embodiment the MTDR is located in the 3′ end of the PTO.

In another embodiment the PTO comprises non-nucleic acid molecules and or nucleic acid analogs.

### Melting Temperature Deciding Region (MTDR)

The length of the MTDR of the PTO which forms the activated Tag Duplex fragment alters the melting temperature of said Tag Duplex fragment. The use of short MTDR regions (e.g. shorter than 10 nucleic acids) will yield a low melting temperature. The inventors have used MTDR of various lengths such as around 16 to around 40 nucleic acids. However the MTDR region of the present invention may comprise 5-100 nucleic acids and/or nucleic acid analogues, such as 10-80, such as 15-70, preferably such as 13-60, more preferably such as 16-39 nucleic acids and/or nucleic acid analogues. Thus an embodiment of the present invention the MTDR comprises 5-50 nucleic acids and/or nucleic acid analogues, such as 10-40, such as 13-30 nucleic acids and/or nucleic acid analogues. When using long MTDR regions (e.g. more than 50 nucleic acids) care should be taken to avoid secondary structures forming within the MTDR itself. In a preferred embodiment the MTDR region of the present invention comprises 13-25 nucleic acids and/or nucleic acid analogues such as locked nucleic acids (LNA). Specific examples of nucleic acid analogs also include, but are not limited to, the following bases in base pair combinations: iso-C/iso-G, iso-dC/iso-dG.

In an embodiment, step (a) comprises hybridizing the target nucleic acid sequence with a PTO; the PTO comprising (i) a targeting portion comprising a hybridizing nucleotide sequence substantially complementary to the target nucleic acid sequence, and (ii) a MTDR, comprising a nucleotide sequence non-complementary to the target nucleic acid sequence, wherein the MDTR comprises 5-50 nucleic acids and/or nucleic acid analogues, such as 10-40, such as 13-30 nucleic acids and/or nucleic acid analogues, and (iii) at least one set of interactive labels comprising at least one fluorophore and at least one quencher; wherein the targeting portion of the PTO is configured to hybridize with the target nucleic acid sequence and MTDR of the PTO is not configured to hybridize with the target nucleic acid sequence.

One advantage of the present invention is that one CQO may detect multiple target sequences identified by unique PTOs. For each target nucleic acid sequence to be detected a PTO with an MTDR sequence is designed which is unique for PTOs having same or similar fluorescent labels, these different PTOs may be referred to as a PTO group if they contain similar and/or identical fluorescent labels. Each PTO group may be detected by a single CQO, which forms an activated Tag Duplex fragment, wherein the activated Tag Duplex fragments are distinguished based on differences in melting temperature as a consequence of the unique MTDR on each PTO in the group. Consequently the number of target nucleic acid sequences which can be detected using only a single fluorescent label is increased.

In another embodiment, for each distinguishable fluorescent label, the method described herein can distinguish at least one, such as at least two, such as at least three, such as at least four, such as at least five, such as at least ten target nucleic acid sequences from each other based on the difference in melting temperature of their respective Tag Duplex fragments, wherein the melting temperature of each Tag Duplex fragment is determined by the length and composition of the MTDR. In another embodiment the length and/or the composition of the MTDR as described herein determines the melting temperature of the activated Tag Duplex fragment described herein above. The melting temperature of the activated Tag Duplex fragment may be any temperature; however a temperature above room temperature is preferable. PCR reactions are conducted in aqueous buffers which typically have a boiling point near 100° C. Thus in an embodiment the melting temperature of the activated Tag Duplex fragment described herein is between 30° C. to 100° C., such as between 35° C. to 90° C., such as between 40° C. to 75° C. such as between 45° C. to 75° C. In a preferred embodiment the melting temperature of the activated Tag Duplex fragment is between 35° C. to 90° C., such as between 50° C. to 85° C. In a further embodiment the MTDRs of the PTO forming Tag Duplex fragment is configured to yield a melting temperature of the activated Tag Duplex fragment between 30° C. to 100° C., such as between 35° C. to 80° C., such as between 40° C. to 75° C. such as between 45° C. to 75° C. In a preferred embodiment the MTDRs of the PTO forming Tag Duplex fragment is configured to yield a melting temperature between 50° C. to 75° C., such as between 50° C. to 70° C. The MTDRs of a group of PTOs are preferably selected so their respective melting temperatures are easily detected and registered. Thus the MTDRs of a group of PTOs may differ in their respective melting temperatures by 2, 3, 4, 5, 6, 7, 8, 9, 10 or more degrees.

In an embodiment step (a) comprises hybridizing the target nucleic acid sequence with a PTO; the PTO comprising (i) a targeting portion comprising a nucleotide sequence substantially complementary to the target nucleic acid sequence, and (ii) an MTDR, comprising a nucleotide sequence non-complementary to the target nucleic acid sequence, wherein the MDTR is configured to yield a melting temperature between 50° C. to 75° C., such as between 50° C. to 70° C., and (iii) at least one set of interactive labels comprising at least one fluorophore and at least one quencher; wherein the targeting portion of the PTO is configured to hybridize with the target nucleic acid sequence and the MTDR of the PTO is not configured to hybridize with the target nucleic acid sequence.

### Linker Molecule

The PTO may further comprise a linker molecule, which is non-complementary to the target nucleic acid sequence and the CQO, between the targeting portion and the MTDR of the PTO. Thus in an embodiment the PTO as described herein further comprises a linker molecule between the targeting portion and the MTDR of the PTO. In some embodiments, the linker is a linker which is non-complementary to the target nucleic acid sequence and the CQO and wherein the linker molecule comprises 1-200 nucleotides, such as 1-50 nucleotides, such as 1-30 nucleotides, such as 2-20 nucleotides, such as about 4-14 nucleotides, such as 6-13 nucleotides, such as 8-12 nucleotides, such as 9-12 nucleotides, such as 11 nucleotides. The linker molecule may comprise or consist of non-nucleic acids such as non-natural or other organic compounds such as carbon chains such as C1-C40 alkanes such as a C6 carbon chain. In an embodiment the linker comprises a mixture of nucleic acids and non-nucleic acids. In another embodiment the linker comprises any organic compound. The linker may be a glycol linker, or any linker known to the person skilled in the art. An advantage of a non-nucleic acid may be that such linkers stop progression of the polymerase along the PTO.

### Blocking Group

Preferably, the 3′-end of the PTO and/or the CQO is “blocked” to prohibit its extension during the PCR reaction. The blocking may be achieved in accordance with conventional methods. For instance, the blocking may be performed by adding to the 3-hydroxyl group of the last nucleotide a chemical moiety such as biotin, a phosphate group, alkyl group, non-nucleotide linker, phosphorothioate and/or an alkane-diol. Alternatively, the blocking may be carried out by removing the 3-hydroxyl group of the last nucleotide or using a nucleotide with no 3-hydroxyl group such as dideoxynucleotide. Thus in an embodiment the PTO and/or CQO further comprises a blocking group in the 3′ end. In another embodiment said blocking group is selected from the group consisting of biotin, a phosphate group, alkyl group, non-nucleotide linker, a phosphorothioate, and/or an alkane-diol. In another embodiment extension of the 3′ end of the PTO and/or CQO is prohibited by removing the 3-hydroxyl group of the last nucleotide of the PTO and/or CQO or by using a nucleotide with no 3-hydroxyl group such as dideoxynucleotide. The quencher on the CQO may act as a blocking group. In an embodiment said blocking group on the CQO is a quencher. In another embodiment said blocking group on the CQO is a quencher located in the 3′ end of said CQO.

The PTOs may be synthesized by click chemistry. A specific MTDR may be used for multiple assays whereas the targeting portion of the PTO varies dependent on the target to be measured. These two elements and the optional linker may thus be joined by click chemistry as is known to those skilled in the art; see also Nucleic Acids Symp Ser (2008) 52(1): 47-48.

### CQO

In an embodiment of the present invention each CQO is used in the present method in the determination of at least one, such as at least two, three, four, five, six, seven, eight, or nine, such as at least ten target nucleic acid sequences, such as 15, 20, 25, 30, 35, 40, 45, 50 or more than 50 target nucleic acids. In a further embodiment two CQOs are used to identify a multitude of target sequences wherein each CQO is used to identify at least one, such as at least two, three, four, five, such as at least ten or more target nucleic acid sequences. In a further embodiment three CQOs, such as at least four, five, six, seven, such as at least eight CQOs are used to identify a multitude of target sequences wherein each CQO is used to identify at least one, such as at least two, three, four, five, such as at least ten target nucleic acid sequences.

In an embodiment a CQO and optionally an enzyme having nuclease activity are in a liquid suspension or liquid solution. In an embodiment at least one CQO and optionally the enzyme having nuclease activity are in a liquid suspension or liquid solution which is ready to use. By ready to use is implied that all conditions for running a PCR reaction are met, i.e. the required salts, pH and so forth are present. In an embodiment at least one CQO and optionally the enzyme having nuclease activity are in a ready to use pellet. In an embodiment at least one PTO and at least one CQO and optionally the enzyme having nuclease activity of the present invention are in a liquid suspension or liquid solution. In an embodiment the at least one PTO and the at least one CQO and optionally an enzyme having nuclease activity of the present invention are in a liquid suspension or liquid solution which is ready to use. In a further embodiment the at least one PTO and the at least one CQO and optionally the enzyme having nuclease activity are in a ready to use pellet. In an embodiment the ready to use pellet comprises a substantially water free composition including e.g. salts and/or nucleotides for running a PCR reaction.

In another embodiment the CQO comprises non-nucleic acid molecules.

In an embodiment the present invention relates to an oligonucleotide i.e. a CQO comprising at least one quencher and a capturing portion, wherein the capturing portion of the CQO is configured to hybridize to at least one, such as two or more PTOs of the present invention, wherein a single CQO may be used in the detection of a multitude of target nucleic acids sequences. A CQO hybridizes to the MTDR region of a PTO. In an embodiment the CQO does not hybridize to the targeting portion of the PTO and/or to the optional linker of the PTO.

The CQO is for use according to the present assays.

### PTO and CQO Fusion

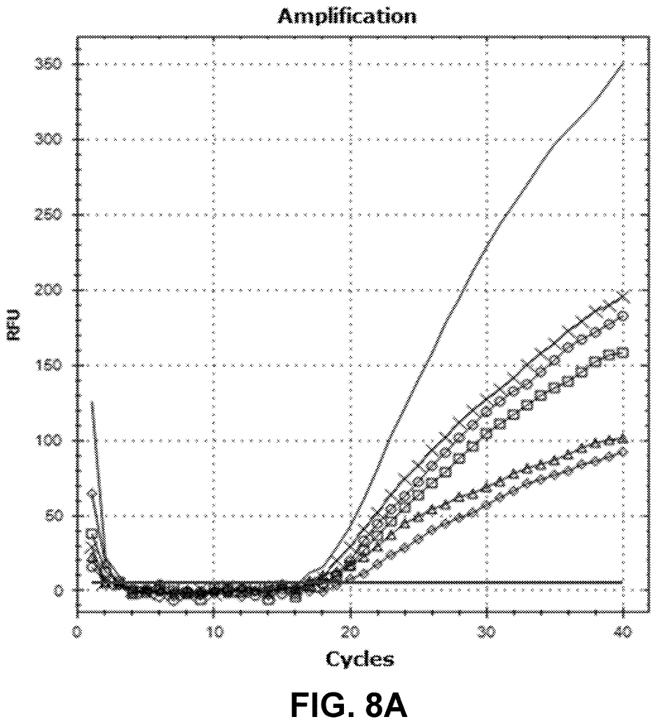

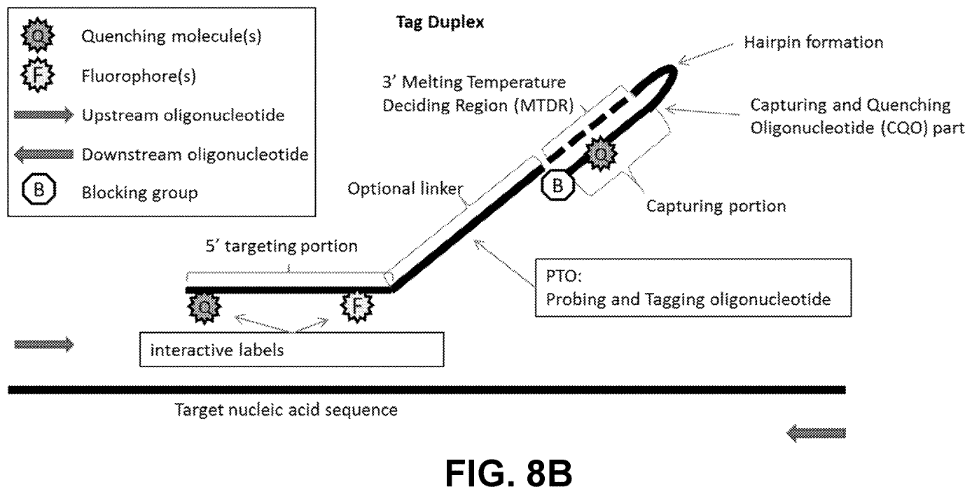

The PTO and the CQO may be linked and configured to reversibly form a hairpin structure wherein the MTDR of the PTO and the capturing portion of the CQO hybridize and thus yield the hairpin structure. FIG. 8A illustrates an example of how the PTO and CQO may yield a hairpin structure. In an embodiment the PTO and the CQO are present on the same oligonucleotide. In another embodiment the PTO and the CQO are linked. In another embodiment the PTO and the CQO are situated on the same oligonucleotide, wherein the MTDR of the PTO and the capturing portion of the CQO are configured to reversibly form a hairpin structure.

The total length of each of the PTO and/or the CQO may vary. In one embodiment the total length of the PTO is between 10 and 500 nucleotides, such as between 20 and 100, such as between 30 and 70 nucleotides. In another embodiment the total length of the CQO is between 10 and 500 nucleotides or base pairs, such as between 15 and 100, such as between 20 and 50 nucleotides or base pairs. In the event the PTO and CQO are fused or linked the total length of the fusion product may be between 20 and 600 nucleotides. In another embodiment the PTO and/or CQO comprises non-natural bases. Examples of artificial nucleic acids (or Xeno Nucleic Acids, XNA) include but are not limited to PNA, LNA, GNA and TNA. These compounds and their use are known to the person of skill. Specific examples of non-natural bases also include but are not limited to the following bases in base pair combinations: iso-C/iso-G, iso-dC/iso-dG. In another embodiment the PTO and/or CQO comprises non-nucleic acid molecules.

### Fluorophores and Quenchers

The inventors have shown that the distance between the fluorophore(s) located on the PTO and the quencher(s) located on the CQO affect the background melting curve generation. In one embodiment the distance between the PTO fluorophore and the CQO quencher molecule is between 6 and 60 base pairs, such as between 10 to 35 base pairs, such as 15-25 base pairs. In another embodiment the fluorophore of the PTO and the quencher (the closest quencher) of the CQO are separated by a distance of between 1 and 40 nucleotides or base pairs, such as between 6 to 35, 10 to 30, 15 to 25, such as about 18 nucleotides.

The distance between the at least one fluorophore of the PTO and the closest of the at least one quencher of the CQO is preferably such that the quenching is sufficient to allow differentiation of the signal between an activated Tag Duplex and a melted Tag Duplex where the PTO and the CQO are not hybridized and the signal is not quenched. Thus, some background signal may occur provided that this signal is lower than the true positive signal, thereby allowing discrimination between background signal and positive signal. Likewise, the choice of fluorophore and quencher should also be such that the background signal and positive signal can be discriminated, as the skilled person is aware.

In an embodiment step (b) of the present invention comprises hybridizing the PTO and the CQO; wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex, wherein at least one fluorophore of the PTO and the at least one quencher of the CQO are separated by a distance of between 1 and 40 nucleotides or basepairs, such as between 6 to 35, 10 to 30, 15 to 25, such as about 20 nucleotides.

In an embodiment the at least one set of interactive labels of the present invention comprises a fluorophore and a quencher, wherein the fluorescence emission from said fluorophore is quenched by said quencher. A set of interactive labels is configured to have compatible fluorophores and quenchers. In another embodiment the at least one set of interactive labels comprises one, two, three, four, five, six, seven, or more sets of interactive labels. In a further embodiment at least two groups of PTOs and CQOs are used for detection of at least two target nucleic acid sequences, wherein each group of PTOs and group of CQOs are configured to have compatible fluorophores and quenchers. In another embodiment the main interaction between the at least one set of interactive labels is mediated by fluorescence resonance energy transfer (FRET).

The inventors have also shown that the distance between the interactive set of labels of the PTO comprising at least one fluorophore and at least one quencher also affects the undesirable background melting curve generation. In an embodiment the interactive set of labels of the PTO is separated by a distance between 1 and 40 nucleotides or base pair, such as between 6 and 35, 10-30, 15 to 25, such as about 20 nucleotides. In an embodiment step (a) of the method described herein comprises hybridizing the target nucleic acid sequence with a PTO; the PTO comprising (i) a targeting portion comprising a nucleotide sequence substantially complementary to the target nucleic acid sequence, and (ii) a MTDR, comprising a nucleotide sequence non-complementary to the target nucleic acid sequence, and (iii) at least one set of interactive labels comprising at least one fluorophore and at least one quencher, wherein the at least one fluorophore and at least one quencher are separated by a distance between 3.4 Å and 136 Å, such as between 20.4 Å and 119 Å, 34 Å and 102 Å, 51 Å and 85 Å, such as about 61.2 Å; wherein the targeting portion of the PTO is configured to hybridize with the target nucleic acid sequence and MTDR of the PTO is not configured to hybridize with the target nucleic acid sequence. In an embodiment step (a) of the method described herein comprises hybridizing the target nucleic acid sequence with a PTO; the PTO comprising (i) a targeting portion comprising a nucleotide sequence substantially complementary to the target nucleic acid sequence, and (ii) a MTDR, comprising a nucleotide sequence non-complementary to the target nucleic acid sequence, and (iii) at least one set of interactive labels comprising at least one fluorophore and at least one quencher, wherein the at least one fluorophore and at least one quencher are separated by a distance between 1 and 40 nucleotides or base pair, such as between 6 and 35, 10-30, 15 to 25, such as about 18 nucleotides; wherein the targeting portion of the PTO is configured to hybridize with the target nucleic acid sequence and MTDR of the PTO is not configured to hybridize with the target nucleic acid sequence.

In an embodiment the interactive set of labels of the PTO are placed so emission from the at least one fluorophore of the PTO is quenched by the at least one quencher of the PTO and by the at least one quencher of the CQO when the PTO and CQO are hybridized. In another embodiment the interactive set of labels of the PTO are placed so emission from the at least one fluorophore of the PTO is quenched by the at least one quencher of the PTO and by the at least one quencher of the CQO, wherein the level of quenching of the at least one fluorophore of the PTO by the least one quencher of the PTO and the at least one quencher of the CQO is substantially similar when the PTO and CQO are hybridized. In a preferred embodiment the interactive set of labels of the PTO are placed so emission from the at least one fluorophore of the PTO is quenched by the at least one quencher of the PTO and by the at least one quencher of the CQO, wherein the distance between of the at least one fluorophore of the PTO by the least one quencher of the PTO and the at least one quencher of the CQO is substantially similar when the PTO and CQO are hybridized. In a further embodiment the interactive set of labels of the PTO are placed so the level of quenching of the at least one fluorophore of the PTO by the at least one quencher of the PTO is substantially similar to and/or stronger than the level of quenching by the at least one quencher of the CQO when the PTO and CQO are hybridized. In another embodiment the interactive set of labels of the PTO are placed so the distance from the at least one fluorophore of the PTO and the at least one quencher of the PTO is substantially similar to and/or shorter than the distance from the at least one fluorophore of the PTO to the at least one quencher of the CQO when the PTO and CQO are hybridized.

Fluorophores which may be conjugated to an oligonucleotide may be used in the present invention. In an embodiment the PTO described herein comprises at least one fluorophore. In another embodiment the at least one fluorophore of the PTO is selected from the group comprising 6-carboxyfluorescein (FAM), tetrachlorofluorescein (TET) or a combination hereof. In another embodiment the PTO of the present invention comprises more than one fluorophore, such as two, three, four, five, six, seven, and/or such as eight fluorophores. In an embodiment the PTO of the present invention comprises two or more identical fluorophores, such as three, four, five, six, seven, and/or such as eight identical fluorophores. In another embodiment the PTO of the present invention comprises two or more different fluorophores, such as three, four, five, six, seven, and/or such as eight different fluorophores.

To facilitate quenching of the at least one fluorophore on the PTO as described herein the PTO comprises at least one quencher molecule. Quenchers which may be conjugated to an oligonucleotide may be used in the present invention. The at least one quencher and the at least one fluorophore of the PTO are configured to be at least one set of interactive labels. In an embodiment the PTO described herein comprises at least one quencher. In another embodiment the at least one quencher of the PTO is configured to quench the at least one fluorophore of the PTO. In another embodiment the at least one fluorophore of the PTO is selected from the group comprising black hole quencher (BHQ) 1, BHQ2, and BHQ3, Cosmic Quencher (e.g. from Biosearch Technologies, Novato, USA), Excellent Bioneer Quencher (EBQ) (e.g. from Bioneer, Daejeon, Korea) or a combination hereof. In a further embodiment the PTO of the present invention comprises more than one quencher, such as two, three, four, five, six, seven, and/or such as eight quenchers. In an embodiment the PTO of the present invention comprises two or more identical quenchers, such as three, four, five, six, seven, and/or such as eight identical quenchers. In another embodiment the PTO of the present invention comprises two or more different quenchers, such as three, four, five, six, seven, and/or such as eight different fluorophores.

To facilitate quenching of the at least one fluorophore on the PTO as described herein the CQO comprises at least one quencher molecule. Most quenchers which may be conjugated to an oligonucleotide may be used in the present invention. However, the at least one quencher of the CQO and the at least one fluorophore of the PTO may be configured to be at least one set of interactive labels. In an embodiment the CQO described herein comprises at least one quencher. In another embodiment the at least one quencher of the CQO is configured to quench the at least one fluorophore of the PTO as described herein. In another embodiment the at least one quencher of the CQO is selected from the group comprising black hole quencher (BHQ) 1, BHQ2, and BHQ3 (from Biosearch Technologies, Novato, USA). In a further embodiment the CQO of the present invention comprises more than one quencher, such as two, three, four, five, six, seven, and/or such as eight quenchers. In an embodiment the CQO of the present invention comprises two or more identical quenchers, such as three, four, five, six, seven, and/or such as eight quenchers. In another embodiment the CQO of the present invention comprises two or more different quenchers, such as three, four, five, six, seven, and/or such as eight fluorophores.

A fluorophore which may be useful in the present invention may include any fluorescent molecule known in the art. Examples of fluorophores are: Cy2™ Cfflfi), YO-PRn™-1 (509), YDYO™-1 (509), Calrein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™(531), Calcium Green™ (533), TO-PRO™-1 (533), TOTOl (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™(576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red(615), Nile Red (628), YO-PRO™_3 (631), YOYO™-3 (631), R-phycocyanin (642), C-Phycocyanin (648), TO-PRO™-3 (660), TOT03 (660), DiD DilC(5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Fluorescein-C3 (520), Pulsar 650 (566), Quasar 570 (667), Quasar 670 (705) and Quasar 705 (610). The number in parenthesis is a maximum emission wavelength in nanometers. In a preferred embodiment the fluorophore is selected from the group consisting of FAM and/or TET. It is noteworthy that a non-fluorescent black quencher molecule capable of quenching a fluorescence of a wide range of wavelengths or a specific wavelength may be used in the present invention. In a preferred embodiment the set of interactive labels are FAM/BHQ. Other suitable pairs of fluorophores/quenchers are known in the art.

### Step (a) Hybridizing the PTO to a Target Sequence

Step (a) of the present invention relates hybridization of the PTO of the present invention to the target nucleic acid. In an embodiment step (a) of the method described herein relates to hybridizing the target nucleic acid sequence with a PTO; the PTO comprising (i) a targeting portion comprising a nucleotide sequence substantially complementary to the target nucleic acid sequence, and (ii) a MTDR, comprising a nucleotide sequence non-complementary to the target nucleic acid sequence, and (iii) at least one set of interactive labels comprising at least one fluorophore and at least one quencher; wherein the targeting portion of the PTO is configured to hybridize with the target nucleic acid sequence and MTDR of the PTO is not configured to hybridize with the target nucleic acid sequence.

In an embodiment step (a) comprises hybridizing the target nucleic acid sequence with a PTO; the PTO comprising (i) a targeting portion comprising a nucleotide sequence substantially complementary to the target nucleic acid sequence, and (ii) a MTDR, comprising a nucleotide sequence non-complementary to the target nucleic acid sequence, and (iii) at least one set of interactive labels comprising at least one fluorophore and at least one quencher; wherein the targeting portion of the PTO is configured to hybridize with the target nucleic acid sequence and MTDR of the PTO is not configured to hybridize with the target nucleic acid sequence, wherein the PTO is between 10 and 500 nucleotides, such as between 20 and 100, such as between 30 and 70 nucleotides or base pairs.

The PTOs and CQOs of the present invention may be premixed prior to addition of the target sequence in the present invention. Thus the Tag Duplex formation of step (b) of the present invention may form prior to hybridization of the PTO to the target sequence. In an embodiment step (b) is performed prior to step (a) as follows;
(b) hybridizing a PTO with a CQO (Capturing and Quenching Oligonucleotide), wherein the PTO comprises (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher; wherein the targeting portion of the PTO is configured to hybridize with the target nucleic acid sequence and MTDR of the PTO is not configured to hybridize with the target nucleic acid sequence; wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex; and
(a) hybridizing the target nucleic acid sequence with said Tag Duplex;
(c) contacting the Tag Duplex with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore;
(d) melting and/or hybridizing said activated Tag Duplex fragment to obtain a signal from the at least one fluorophore, and
(e) detecting the activated Tag Duplex fragment by measuring the signal from the at least one fluorophore; wherein the signal is indicative of the presence of the target nucleic acid sequence in the nucleic acid mixture.

It will be understood that in order to obtain a signal from the at least one fluorophore, said activated Tag Duplex fragment does not comprise the at least one quencher comprised in the at least one set of interactive labels of the MTDR. In other words, the signal is obtained when the at least one fluorophore and the at least one quencher of the at least one set of interactive labels no longer interact.

The PTOs, CQOs and target sequences of the present invention may also be mixed simultaneously. In an embodiment step (a) and step (b) of the present invention may be carried out in any order or simultaneously.

### Upstream and Downstream Oligonucleotides

The addition of a pair of PCR primers located upstream and downstream of the binding site of the PTO to the target nucleic acid sequence increases the specificity of PCR assays and assists in avoiding false positive hybridization signals. Thus the present invention may further include an upstream primer which is complementary to the target nucleic acid and a downstream primer which may hybridize downstream of the PTO binding site on the target nucleic acid. The upstream and downstream oligonucleotides are configured not to overlap with the PTO binding site of the target nucleic acid sequence. The upstream and downstream primers may be located within 2000 base pairs of the target nucleic acid sequence. In one embodiment the upstream oligonucleotide and downstream nucleotide are located at least one base pair from the PTO binding site of the target nucleic acid sequence. In another embodiment the upstream and/or downstream oligonucleotides are located between 1-2000 base pairs from the PTO binding site of the target nucleic acid sequence. The upstream and/or downstream oligonucleotides may be located more than 2000 base pairs (2 kb) from the target nucleic acid sequence, such as 2.5 kb, such as 3 kb, such as 3.5 kb, such as 4 kb, such as 5 kb, such as 10 kb, such as 20 kb from the target nucleic acid sequence.

### Decontamination

Decontamination of the reaction vessel may take place prior to step (a). Thus in an embodiment a UNG treatment step (BioTechniques 38:569-575 (April 2005)) and/or a denaturation step is used prior to step (a). RNA decontamination treatments known to the skilled person may be applied. In an embodiment a decontamination treatment step and/or a denaturation step is used prior to step (a).

### Step (b) Hybridizing a CQO to the PTO Forming a Tag Duplex

Step (b) of the present method concerns the hybridization of the PTO and the CQO which forms a Tag Duplex. In an embodiment step (b) of the present method comprises hybridizing the PTO and the CQO; wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex.

As previously described the inventors have shown that the distance between the fluorophore(s) located on the PTO and the quencher(s) located on the CQO affect the background melting curve generation.

In an embodiment step (b) of the present method comprises hybridizing the PTO and the CQO; wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex, wherein the distance between the at least one fluorophore on the PTO and the at least one quenching molecule on the CQO quencher molecule is between 1 and 60 base pairs, such as between 10 to 35 base pairs, such as 15-25 base pairs, such as about 18 base pairs.

In an embodiment step (b) of the present method comprises hybridizing the PTO and the CQO; wherein the CQO comprises (i) a capturing portion comprising a nucleotide sequence which is reverse complementary to the MTDR of the PTO and (ii) at least one quenching molecule configured for quenching of the at least one fluorophore of the PTO; wherein the MTDR of the PTO is configured to hybridize with the capturing portion of the CQO to form a Tag Duplex.

### Step (c) Release of an Activated Tag Duplex Fragment

Step (c) of the present invention relates to nuclease mediated cleavage of the Tag Duplex which forms a released Tag Duplex fragment. In an embodiment step (c) of the present invention comprises contacting the Tag Duplex from step (b) with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore.

To avoid false positive signals the at least one quencher of the CQO is configured to reversibly quench the at least one fluorophore of the PTO when the activated Tag Duplex fragment is hybridized. In an embodiment of the present method the at least one quencher of the CQO is configured to reversibly quench the at least one fluorophore of the activated Tag Duplex fragment. As explained above, the quencher of the CQO and the fluorophore of the PTO should be chosen such that the background signal and the positive signal can be discriminated. Likewise, the distance between the quencher of the CQO and the fluorophore of the PTO may have to be optimized to obtain a desired discrimination of signals.

Any enzyme having nuclease activity may induce the release of the Tag Duplex as shown in FIGS. 1A-1C. In an embodiment the nuclease activity described above is 5′ nuclease activity of a FEN nuclease (flap endonuclease). In another embodiment the enzyme having nuclease activity is a template dependent DNA polymerase such as a thermostable template dependent DNA polymerase, such as a Taq polymerase and/or a Sso7d-fusion polymerase or mixtures thereof. In a preferred embodiment the enzyme having nuclease activity is a Sso7d-fusion polymerase. In another preferred embodiment the enzyme having nuclease activity is the GoTaq polymerase. In another embodiment the enzyme having nuclease activity is a template dependent DNA polymerase having 5′ to 3′ exonuclease activity. Thus in an embodiment the nuclease is an exonuclease.

The skilled person knows that the concentration of polymerase may influence its activity. The optimal concentration may also depend on further parameters such as the concentration of target, template, or the sequence of the nucleic acids present in the reaction. The skilled person knows how to optimize the polymerase concentration in order to achieve good results.

The release of the Tag Duplex, resulting in the formation of the activated Tag Duplex fragment is mediated by the enzyme having nuclease activity upon extension of the upstream oligonucleotide described herein. In an embodiment the cleavage of the PTO part of the Tag Duplex is induced by said template dependent DNA polymerase extending the upstream oligonucleotide, wherein said polymerase has 5′ nuclease activity. In another embodiment the cleavage of the PTO part of the Tag Duplex is induced by said template dependent DNA polymerase upon extension of the upstream oligonucleotide, wherein said polymerase has 5′ nuclease activity.

Tag Duplex fragment is released when the part of the 5′ targeting portion of the PTO is cleaved. The cleavage results in a reduced affinity between the targeting portion of the PTO and the target nucleic acid sequence which consequently results in dissociation of the target nucleic acid sequence and the Tag Duplex thereby forming the activated Tag Duplex fragment. In an embodiment the at least one quencher on the PTO as described herein is released from the PTO by the enzyme having nuclease activity. In another embodiment the enzyme having nuclease activity removes the at least one quencher of the PTO part of the activated Tag Duplex fragment. In another embodiment the activated Tag Duplex fragment comprises a PTO fragment wherein the at least one quencher of the PTO is not present. The at least one quencher may not be present on the activated Tag Duplex fragment as a consequence of the nuclease activity of the enzyme having nuclease activity as described herein and illustrated in FIGS. 1A-1C.

The presence of the activated Tag Duplex and/or activated PTO may be detected by qPCR and/or real time PCR. Preferably only activated Tag Duplex is detected by real time PCR.

### Step (d) Melting the Activated Tag Duplex Fragment

Step (d) of the present invention relates to the melting of the activated Tag Duplex fragment from step (c). In an embodiment step (d) of the present invention comprises melting and/or hybridizing said activated Tag Duplex fragment to obtain a signal from the at least one fluorophore. The temperature at which the melting occurs is registered.

The melting may be carried out by conventional technologies, including, but not limited to, heating, alkali, formamide, urea and glycoxal treatment, enzymatic methods (e.g., helicase action), and binding proteins. For instance, the melting can be achieved by heating at temperature ranging from 30° C. to 100° C.

When the activated Tag Duplex fragment is heated over a range of temperatures the MTDR of the PTO dissociates from the CQO of the present invention. The temperature at which one half of an activated Tag Duplex fragment duplex will dissociate to become single stranded is determined by the stability Tag Duplex which is determined by the melting temperature region (MTDR). Thus in an embodiment the activated Tag Duplex fragment I heated over a range of temperatures. In another embodiment the activated Tag Duplex fragment is heated from 30° C. to 100° C., such as from 35° C. to 100° C., such as from 40° C. to 75° C. such as from 45° C. to 70° C. In a preferred embodiment the melting temperature of the activated Tag Duplex fragment is 45° C. to 70° C. In another embodiment the activated Tag Duplex fragment is melted at a predetermined temperature.

As long as the activated Tag Duplex fragment is double stranded the emission from the at least one fluorophore on the PTO is substantially quenched by the at least one quencher on the CQO. When the activated Tag Duplex fragment dissociates and become single stranded the emission of the at least one fluorophore on the PTO may be unquenched by the at least one quencher on the CQO. Thus in an embodiment the emission from the at least one fluorophore is unquenched when the Tag Duplex is melted in step (d). In another embodiment the emission from the at least one fluorophore is unquenched when the Tag Duplex dissociates and become single stranded in step (d). In another embodiment the presence of an activated Tag Duplex fragment is determined by a melting curve analysis and/or a hybridization curve analysis. In a further embodiment the presence of an activated Tag Duplex fragment is determined by a melting curve analysis and/or a hybridization curve analysis wherein the identified melting temperature of the activated Tag Duplex fragment is determined by the MTDR of the PTO described herein.

Step (e) Detection of Signal from the Melted Tag Duplex Fragment.

Step (e) of the present invention relates to detection of a signal as a consequence of the Tag Duplex melting in step (d). In an embodiment of the present invention step (e) comprises detecting the activated Tag Duplex fragment by measuring the signal from the at least one fluorophore; wherein the signal is indicative of the presence of the target nucleic acid sequence in the nucleic acid mixture.

The nature of the signal to be measured is dependent on the at least one fluorophore on the PTO and may be determined by a range of analytical methods for example real-time PCR detection systems.

A melting curve or hybridization curve may be obtained by conventional technologies. For example, a melting curve or hybridization curve may comprise a graphic plot or display of the variation of the output signal with the parameter of hybridization stringency. The output signal may be plotted directly against the hybridization parameter. Typically, a melting curve or hybridization curve will have the output signal, for example fluorescence, which indicates the degree of duplex structure (i.e. the extent of hybridization), plotted on the Y-axis and the hybridization parameter on the X axis (i.e. the temperature).

For recording a melting curve by fluorescence, typically the overall sample temperature can be either increased or decreased stepwise with set equilibration time of 0-360 s at each temperature. Typically a step size between 0.5° C. and 2° C. is used but it could be lowered to 0.1° C. depending on the desired accuracy. At every temperature, a readout of the fluorescence is recorded for the relevant wavelength. Based on the melting curve, the TM of a DNA duplex under the conditions applied can be determined.

The method of choice for nucleic acid (DNA, RNA) quantification in all areas of molecular biology is real-time PCR or quantitative PCR (qPCR). The method is so-called because the amplification of DNA with a polymerase chain reaction (PCR) is monitored in real time (qPCR cyclers constantly scan qPCR plates).

Fluorescent reporter probes (Tagman probes or dual-labeled probes) detect only the DNA containing the sequence complementary to the probe; therefore, use of the reporter probe significantly increases specificity, and enables performing the technique even in the presence of other dsDNA. Using different-coloured labels, fluorescent probes can be used in multiplex assays for monitoring several target sequences in the same tube. The method relies on a DNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter. The PCR is prepared as normal and the reporter probe is added. As the reaction commences, during the annealing stage of the PCR both probe and primers anneal to the DNA target. Polymerisation of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a real-time PCR machine, and its geometric increase corresponding to exponential increase of the product is used to determine the quantification cycle (Cq) in each reaction.

The signal can be measured either at the temperature of annealing or at the temperature of denaturation of the duplexes present in the reaction. Accordingly, the signal can be measured at a temperature of between 55 and 65° C., such as between 56 and 64° C., such as between 57 and 63° C., such as between 58 and 62° C., such as between 59 and 61° C., such as at 60° C. In other embodiments, the signal can be measured at a temperature of between 9° and 100° C., such as between 91 and 99° C., such as between 92 and 98° C., such as between 94 and 97° C., such as between 95 and 96° C., such as at 95° C. The skilled person knows how to determine which temperature provides the best readout signal.

As shown in example 5, the concentration of target influences the strength of the signal to be detected. Thus, target concentration may be adjusted to improve the signal if needed.

### Target Nucleic Acid Sequences

The target nucleic acid sequence as used herein refers to any sequence which is desirable to identify in a mixture of nucleic acid sequences. The simple assay of the present invention has a multitude of applications. A non-exhaustive list of applications of the assay of the present invention may be:
Human and/or veterinary diagnostics
Food and/or feed quality and safety
Environmental surveillance
Scientific research

Thus in an embodiment the target nucleic acid sequence of the present invention is from a pathogenic organism such as a bacterium, virus, fungus, and/or protozoan. In another embodiment the target nucleic acid sequence of the present invention is from a pathogenic organism capable of infecting a farm animal such as a cow, chicken, pig, horse, sheep, and/or goat. In a preferred embodiment the target nucleic acid sequence of the present invention is from a pathogenic organism capable of infecting a mammal such as a human being, cow, pig, horse, sheep, and/or goat. In a more preferred embodiment the target nucleic acid sequence of the present invention is from a pathogenic organism capable of infecting a human being.

In an embodiment of the present invention the target nucleic acid sequence of the present invention is from a virus capable of infecting a human being. In a further embodiment of the present invention the target nucleic acid sequence of the present invention is from a virus capable of infecting a human being which causes a mortality rate higher than 10%. In an embodiment the virus is an Ebola virus.

In an embodiment of the present invention the target nucleic acid sequence of the present invention is from bacteria capable of infecting a human being. In another embodiment of the present invention the target nucleic acid sequence of the present invention is from bacteria capable of infecting a human being which causes a mortality rate higher than 10%.

In an embodiment of the present invention the target nucleic acid sequence of the present invention is from a pathogenic organism causing a sexually transmitted disease selected from the group consisting of Chlamydia, Gonorrhea, and Herpes.

In an embodiment of the present invention the target nucleic acid sequence of the present invention is from a pathogenic organism selected from the group comprising Methicillin Resistant Staphylococcus Aureus (MRSA).

### Kit for Detection of Target Nucleic Acid Sequences

The elements the present invention may be comprised within a kit of parts.

Thus an aspect of the present invention relates to a kit of parts for detection of a target nucleic acid sequence, the kit comprising:
i. optionally at least one PTO as described herein, and
ii. at least one CQO described herein, and
iii. optionally an enzyme having nuclease activity, and
iv. optionally instructions on how to detect a target nucleic acid sequence.

The kit may be used for detection of more than one target nucleic acid sequences. In an embodiment the kit described herein further comprises:
i. optionally at least two PTOs from at least two different groups of PTOs, and
ii. at least two CQOs.

The kit may also contain at least one downstream and/or upstream oligonucleotide as described herein. In an embodiment the kit further comprises a downstream oligonucleotide and/or an upstream oligonucleotide as described herein.

Additionally the kit may further comprise an enzyme with nuclease activity. Thus in an embodiment the kit further comprises an enzyme with nuclease activity as described herein.

In an embodiment the kit described herein is a liquid suspension or liquid solution. In an embodiment the kit described above is a liquid suspension or liquid solution which is ready to use. In a further embodiment the described kit is in a ready to use pellet. In an embodiment the ready to use pellet comprises a substantially water free composition comprising i), ii), and iii) of said kit.

In an embodiment the kit described herein comprises PTOs and CQOs which are partially and/or fully hybridized.

The kit may also include at least one Tag Duplex which may be used as control and for Tₘ calibration of e.g. the applied analytical equipment. The Tₘ of an oligonucleotide may vary depending on e.g. the salt concentration, DNA concentration, pH and the presence of denaturants (such as formamide or DMSO). Such control may be desirable if the samples to be analyzed contain varying i.e. salt concentrations. In an embodiment the kit described herein further comprises a control sample and/or control Tag Duplex.

### Diagnosis

The present methods may be used in the diagnosis and/or treatment of individuals in need thereof.

Thus an embodiment of the present invention relates to a method of diagnosing an individual as having a disease or disorder characterized by the presence of a target nucleic acid sequence said method comprising the steps of the assay as indicated elsewhere and resulting in the detection of said target nucleic acid sequence.

A non-limiting example hereof is: A method of diagnosing an individual as having a disease or disorder characterized by the presence of a target nucleic acid sequence said method comprising the steps of:
Step (a) hybridizing a target nucleic acid sequence with a PTO (Probing and Tagging Oligonucleotide); the PTO comprising (i) a targeting portion comprising a nucleotide sequence substantially complementary to the target nucleic acid sequence, and (ii) a Melting Temperature Deciding Region (MTDR), comprising a nucleotide sequence non-complementary to the target nucleic acid sequence, and (iii) at least one set of interactive labels comprising at least one fluorophore and at least one quencher;
Step (b) hybridizing said PTO with a CQO (Capturing and Quenching Oligonucleotide); wherein the CQO comprises (i) a capturing portion comprising a nucleotide sequence which is reverse complementary to the MTDR of the PTO and (ii) at least one quenching molecule; wherein the MTDR of the PTO is configured to hybridize with the capturing portion of the CQO to form a Tag Duplex;
Step (c) contacting the Tag Duplex with an enzyme having nuclease activity; wherein the enzyme having nuclease activity induces cleavage of the Tag Duplex when the Tag Duplex is hybridized with the target nucleic acid sequence thereby releasing an activated Tag Duplex fragment comprising a PTO fragment comprising the MTDR hybridized to the capturing portion of the CQO and the at least one fluorophore; Step (d) melting and/or hybridizing said activated Tag Duplex fragment to obtain a signal from the at least one fluorophore, and
Step (e) detecting the activated Tag Duplex fragment by measuring the signal from the at least one fluorophore; wherein the signal is indicative of the presence of the target nucleic acid sequence and thus the presence of the disease or disorder in said individual.

The disease or disorder may be an infection caused i.e. by a pathogen or be a genetic disorder or disease.

### Reaction Mixture

In a further embodiment the invention comprises a reaction mixture. Thus an embodiment of the invention provides a reaction mixture for use in a process for the amplification and/or detection of a target nucleic acid sequence in a sample wherein the reaction mixture, prior to amplification, comprises at least one pair of oligonucleotide primers, at least one PTO and at least one CQO, wherein said pair of primers, PTO and CQO are characterized in that said pair of oligonucleotide primers comprises a first a primer complementary to said target nucleic acid and which primes the synthesis of a first extension product that is complementary to said target nucleic acid, and a second primer complementary to said first extension product and which primes the synthesis of a second extension product; and said PTO hybridizes to a nucleotide sequence substantially complementary to the target nucleic acid sequence or the complement of said target nucleic acid, wherein said region is between one member of said primer pair and the complement of the other member of said primer pair and the PTO comprises at least one set of interactive labels, a MTDR, and optionally a linker between the targeting portion and the MTDR; and wherein the CQO comprises at least one quencher and a capturing portion, said capturing portion being configured to hybridize to the PTO. The reaction mixture may comprise several oligonucleotide primer pairs, several PTOs and a single CQO. The reaction mixture may comprise a single CQO configured to hybridize to all PTOs in the reaction mixture.

### Computer Implemented Method

Another aspect of the present invention relates to a computer-implemented method for identifying at least one target sequence, the method comprising the steps of 1) providing information about PTOs, CQOs, target sequences, and 2) obtaining signals from at least one melted Tag Duplex fragment, and 3) identification of at least one target sequence on the basis of said provided information and obtained signals.

### EXAMPLES

### Example 1 Distance Between the Fluorophore and the CQO Quencher

The following example shows results of a PCR reaction comprising 5 different designs of tagging probes and a TaqMan probe specific for the ipaH gene. All reactions are performed with the 2 common primers (DEC229F and DEC230R). Tagging probes were designed such that FAM is inserted furthest from the location of quenchers in the hybridised targeting portion of the PTO in DEC486P and closest to the location of quenchers in the hybridized targeting portion of the PTO in DEC490P. The five different tagging probe designs were tested in combination with a single CQO design (DEC481rP). The results illustrate that by increasing the distance between FAM and the location of quenchers in the hybridized targeting portion of the PTO, background melting curve generation is reduced. It further illustrates that the preferred PTO design (DEC486P) generates PCR amplification curves only in the presence of the specific target, and also only generates melting curve signal in the presence of amplified target. The other designs included (DEC487P-490P) all show background melting curves in the NTC reaction indicating false positive results. The TaqMan probe DEC464 is included as a positive control for the PCR reaction.

TABLE 1
Primer and probes sequences (5′ to 3′):

| | | Seq |
|---|---|---|
| | | ID |
| | | NO: |
| PCR | | |
| Primers: | | |
| DEC229F | ZGTCCATCAGGCATCAGAAGG | 1 |
| DEC230R | ZGGTAGACTTCTATCTCATCCAC | 2 |
| CQO | | |
| (Quenching | | |
| probe) | | |
| DEC481rP | GATACTAGAGTTTCATAGTTCGTAGTCAAGAT | 3 |
| | GATAGATTGGAAGTGCG-(dT-BHQ1)-CAG- | |
| | (dT-BHQ2)-CAG-BHQ3 | |
| Tagging | | |
| Probe | | |
| (PTO) | | |
| DEC486P | BHQ1-AATGTTCCGCC(dT-FAM)CGAAATT | 4 |
| | CTGGAGTATATCGACTGACGCACTTCCAA-phos | |
| DEC487P | AATGT(dT-BHQ1)CCGCCTCGAAA(dT-FAM) | 5 |
| | TCTGGAGTATATCGACTGACGCACTTCCAA-phos | |
| DEC488P | BHQ1-AATGTTCCGCCTCGAAA(dT-FAM) | 6 |
| | TCTGGAGTATATCGACTGACGCACTTCCAA-phos | |
| DEC489P | BHQ1-AATGTTCCGCCTCGAAATTC(dT-FAM) | 7 |
| | GGAGTATATCGACTGACGCACTTCCAA-phos | |
| DEC490P | BHQ1-AATGTTCCGCCTCGAAATTCTGGAG(dT- | 8 |
| | FAM)ATATCGACTGACGCACTTCCAA-phos | |
| TaqMan | | |
| Probe | | |
| DEC464 | FAM-AATGTTCCGCCTCGAAATTCTGGAG-BHQ1 | 9 |

Z = TINA, BHQ1 = black hole quencher 1, BHQ2 = black hole quencher 2, BHQ3 = black hole quencher 3, (dT-FAM) = internal FAM attached to T. (dT-BHQ1) = internal BHQ1 attached to T. (dT-BHQ2) = internal BHQ2 attached to T.

### PCR Reaction:

A 10 μL reaction was prepared that contained 1×SsoAdvanced Universal Probes Supermix (prod. #172-5280, Bio-Rad), 200 nM forward and reverse primer, 400 nM tagging probe, 800 nM quenching probe, 1:200 dilution of target, 0.25 U Uracil DNA Glycosylase, 1 U/μL, (#EN0361 Fermentas). Target was prepared by mixing a single colony of E. coli in 200 μl water and boiling 95° C. for 15 minutes. 25 μL boiled target was subsequently added to 100 μL sterile water as a target stock solution which was diluted 5× in the final PCR reaction. Reactions were assembled in AB gene SuperPlate 96-well PCR plate (kat.nr. AB2800) and sealed with Optically clear, adhesive, Microseal B film (Bio-Rad, Cat.nr. MSB 1001).

PCR reaction mix was subjected to the following PCR cycling and melting curve program (Bio-Rad CFX96 Real-Time PCR Detection System):

PCR program:

| | Temp (° C.) | Time | |
|---|---|---|---|
| 1 | 40 | for 10 minutes | UNG treatment |
| 2 | 95 | for 10 minutes | Activation/denaturation |
| 3 | 95 | for 15 seconds | a) Denaturation |

Plate Read

| 4 | 60 | for 60 seconds | b) Annealing/elongation |
|---|---|---|---|
| | GoTo 3, 39 more times | | |
| 5 | 95 | for 10 seconds | |
| 6 | Melt Curve 40° C. to 95° C., increment 0.5° C. for 5 seconds, | | |
| 7 | 10 | for 10 minutes | |

End

### Example 2 Multiplex PCR Reaction

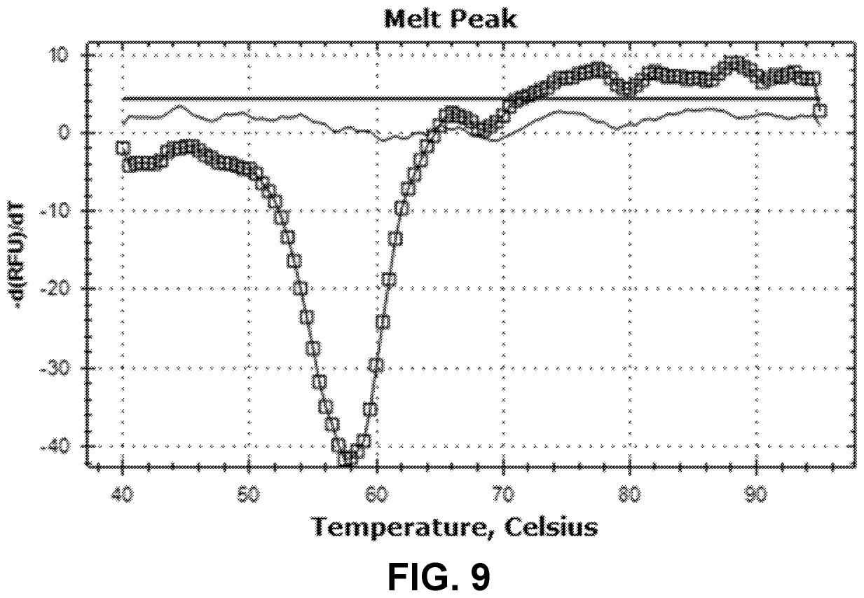

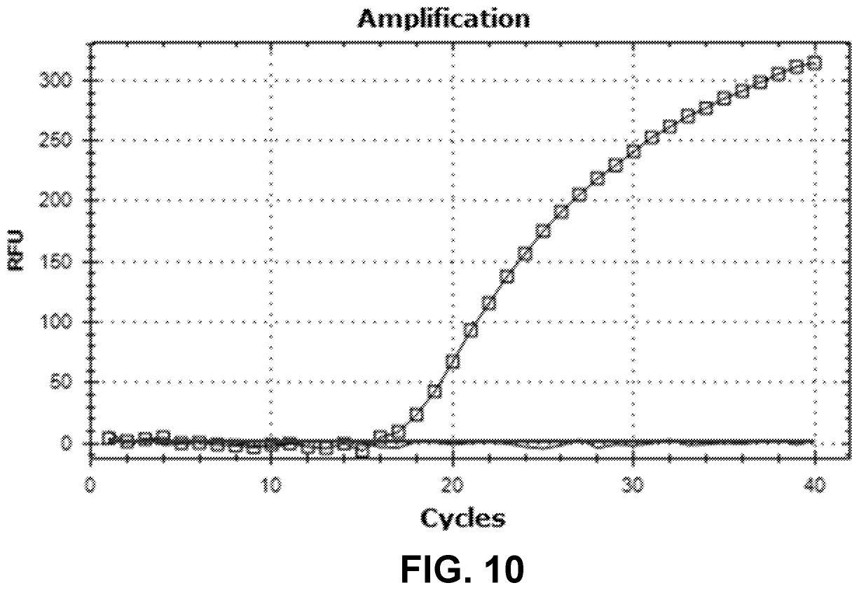

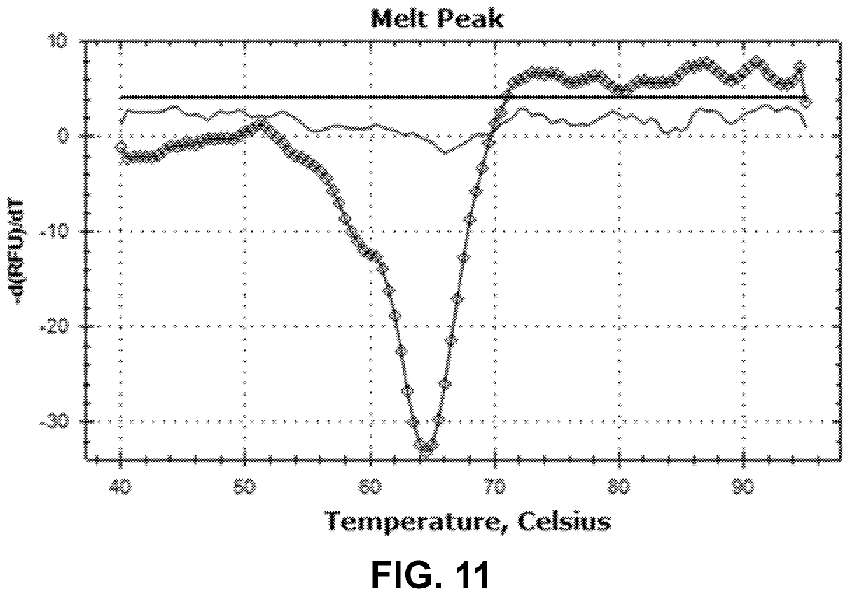

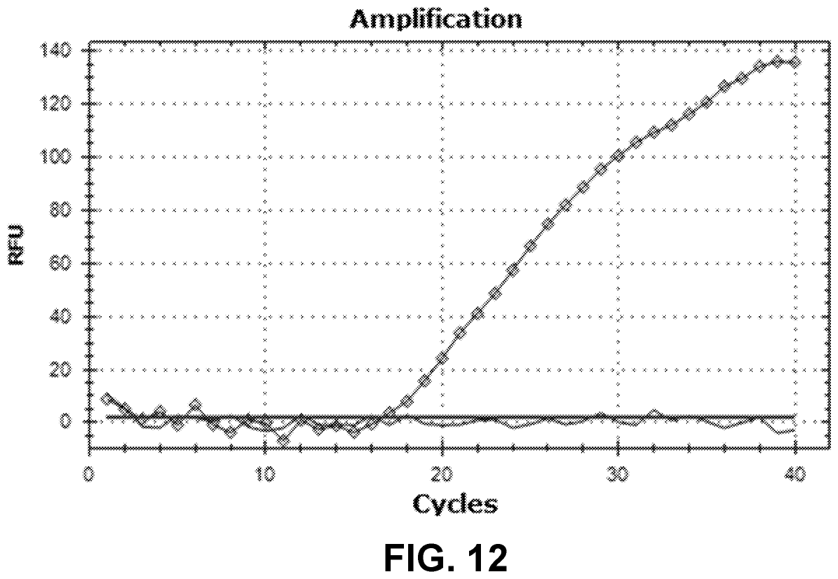

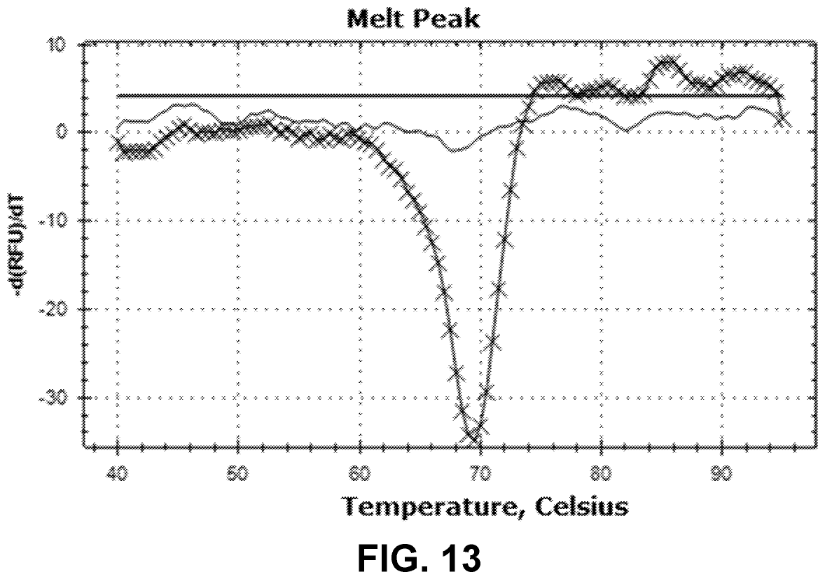

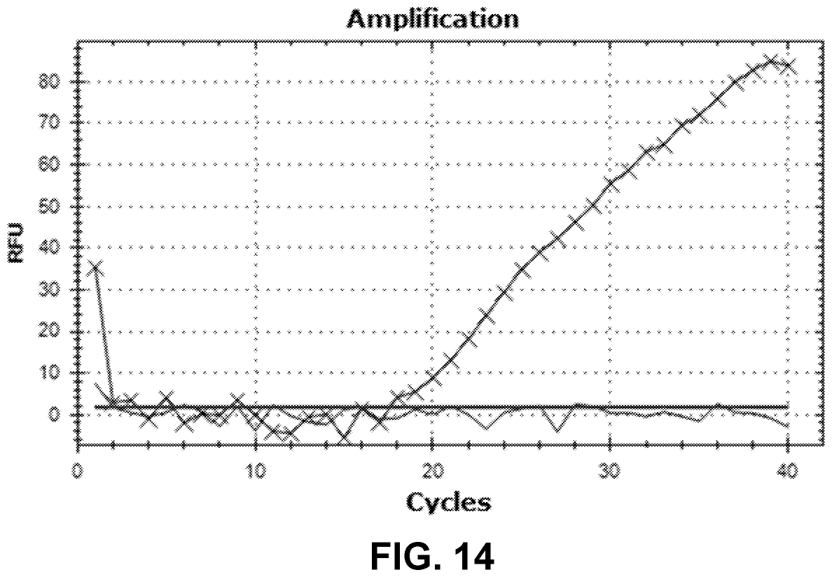

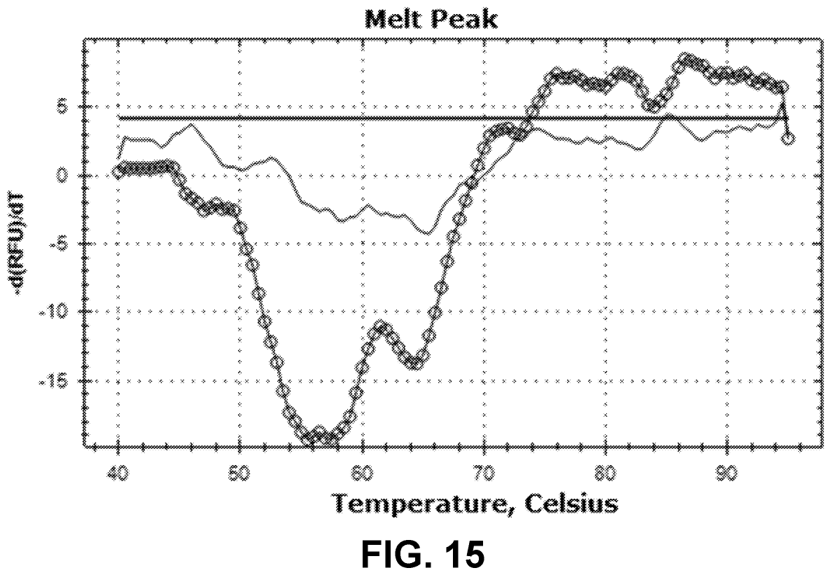

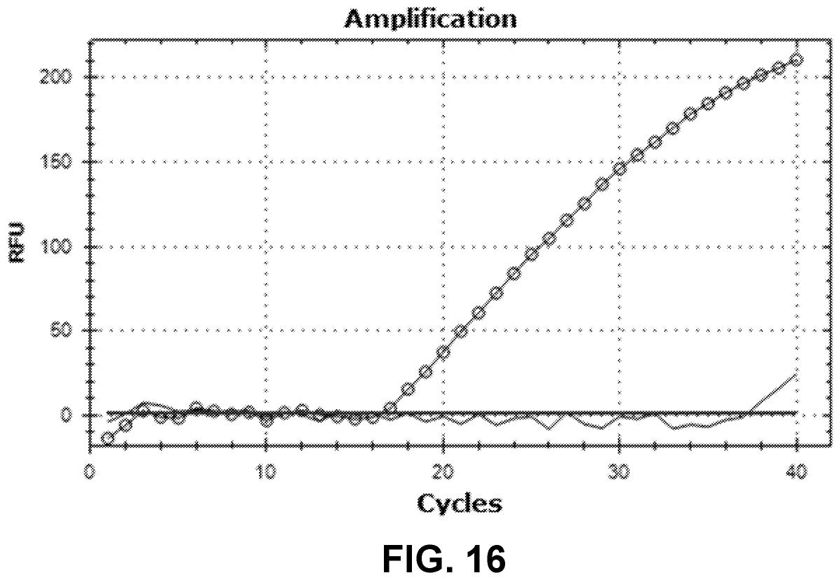

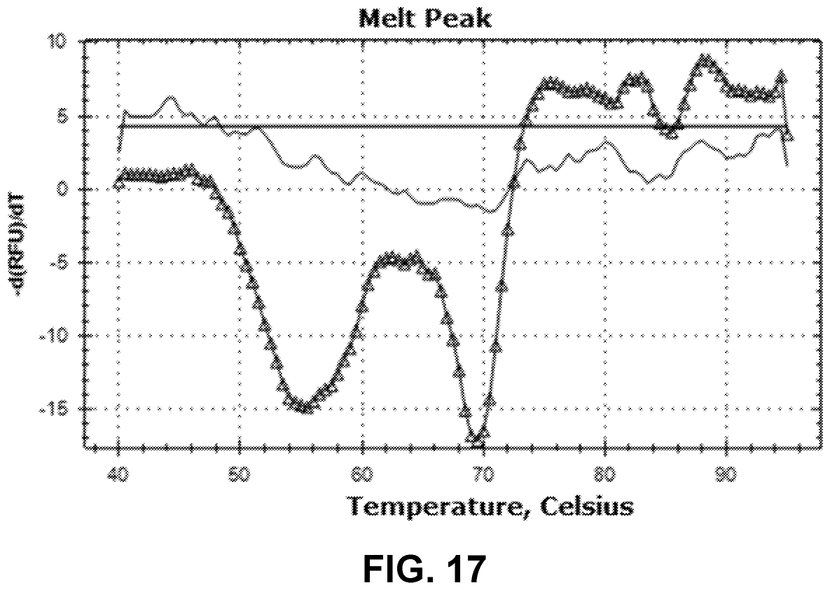

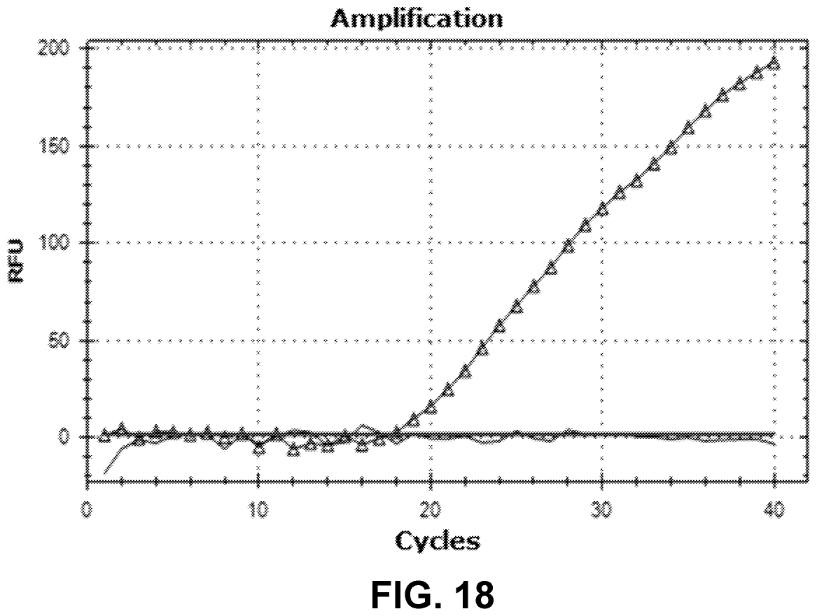

The following example shows results of PCR reactions comprising 3 different designs of PTO (tagging probes) specific for the ipaH gene, carrying increasing length of MTDR region where DEC500P has the shortest and thereby lowest Tₘ, and DEC503P has the longest MTDR and hence highest Tₘ. All reactions were performed with the 2 common primers (DEC229F and DEC230R). The 3 different tagging probes were tested in combination with a single CQO (quenching probe) design (DEC481rP). The 3 PTO designs were tested alone (FIGS. 9-14), combining probes DEC500P with DEC502P (FIGS. 15-16), and combining probes DEC500P and DEC503P (FIGS. 17-18). The results illustrate that each of the PTO perform well in PCR alone and in combination. In particular, the results furthermore show that combining probes DEC500P with DEC502P provides 2 individually distinguishable melting curves, indicating that both probes detect the specific signal. The results furthermore show, that combining probes DEC500P with DEC503P also provides 2 individually distinguishable melting curves with even wider spaced curves in accordance with the bigger Tm difference between the MTDR of DEC500P and DEC503P while still showing that both probes detect the specific signal. The results further show that all probes provide very low background melting curves in the NTC reaction indicating that the PTO probes do not provide a signal without the presence of the specific target.

TABLE 2
Primer and probes sequences (5′ to 3′):

| | Seq | |
|---|---|---|
| | ID NO: | |
| PCR | | |
| Primers: | | |
| DEC229F | 1 | ZGTCCATCAGGCATCAGAAGG |
| DEC230R | 2 | ZGGTAGACTTCTATCTCATCCAC |
| Quenching | | |
| probe | | |
| DEC481rP | 3 | GATACTAGAGTTTCATAGTTCGTAGTCAAG |
| | | ATGATAGATTGGAAGTGCG-(dT-BHQ1)- |
| | | CAG-(dT-BHQ2)-CAG-BHQ3 |
| Tagging | | |
| Probe | | |
| DEC500P | 10 | BHQ1-AATGTTCCGCC(dT-FAM)CGAAAT |
| | | TCTGGAGTATA |
| | | CTGACTGACGCACTTCCAA-phos |
| DEC502P | 11 | BHQ1-AATGTTCCGCC(dT-FAM)CGAA |
| | | ATTCTGGAGTATA |
| | | CTGACTGACGCACTTCCAATCTATCATC-phos |
| DEC503P | 12 | BHQ1-AATGTTCCGCC(dT-FAM)CGAAATTCT |
| | | GGAGTATACTGACTGACGCACTTCCAATCTATC |
| | | ATCTTGACTACG-phos |

Z = TINA, BHQ1 = black hole quencher 1, BHQ2 = black hole quencher 2, BHQ3 = black hole quencher 3, (dT-FAM) = internal FAM attached to T. (dT-BHQ1) = internal BHQ1 attached to T. (dT-BHQ2) = internal BHQ2 attached to T. phos = phosphate group to block extension.

### PCR Reaction:

A 10 ul reaction was prepared that contained 1×GoTaq Probe qPCR mastermix (prod. #Promega A6101), 200 nM forward and reverse primer, 400 nM tagging probe, 800 nM quenching probe, 1:200 dilution of target, 0.25 U Uracil DNA Glycosylase, 1 U/μL, (#EN0361 Fermentas). Target was prepared by mixing a single colony of E. coli carrying the ipaH gene in 200 ul water and boiling 95° C. for 15 minutes. 25 ul boiled target was subsequently added to 100 ul sterile water as a target stock solution which was diluted 5× in the final PCR reaction. Reactions were assembled in AB gene SuperPlate 96-well PCR plate (kat.nr. AB2800) and sealed with Optically clear, adhesive, Microseal B film (Bio-Rad, Cat.nr. MSB1001).

PCR reaction mix was subjected to the following PCR cycling and melting curve program (Bio-Rad CFX96 Real-Time PCR Detection System):

| | Temp (° C.) | Time | |
|---|---|---|---|
| 1 | 40 | For 10 minutes | UNG treatment |
| 2 | 95 | For 10 minutes | Activation/denaturation |
| 3 | 95 | For 15 seconds | a) Denaturation |

Plate Read

| 4 | 60 | For 60 seconds | b) Annealing/elongation |
|---|---|---|---|
| | GoTo 3, 39 more times | | |
| 5 | 95 | for 10 seconds | |
| 6 | Melt Curve 40° C. to 95° C., increment 0.5° C. for 5 seconds | | |
| 7 | 10 | For 10 minutes | |

End

### Example 3 Loop Probe Design

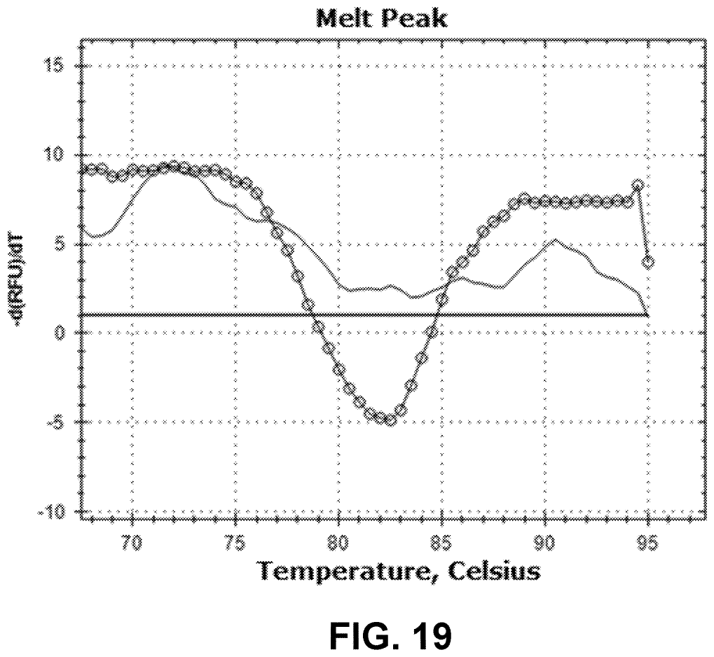

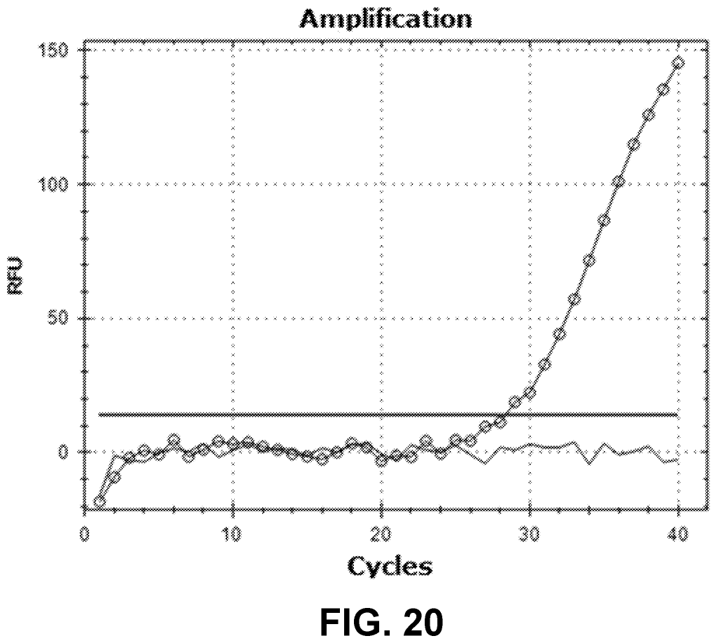

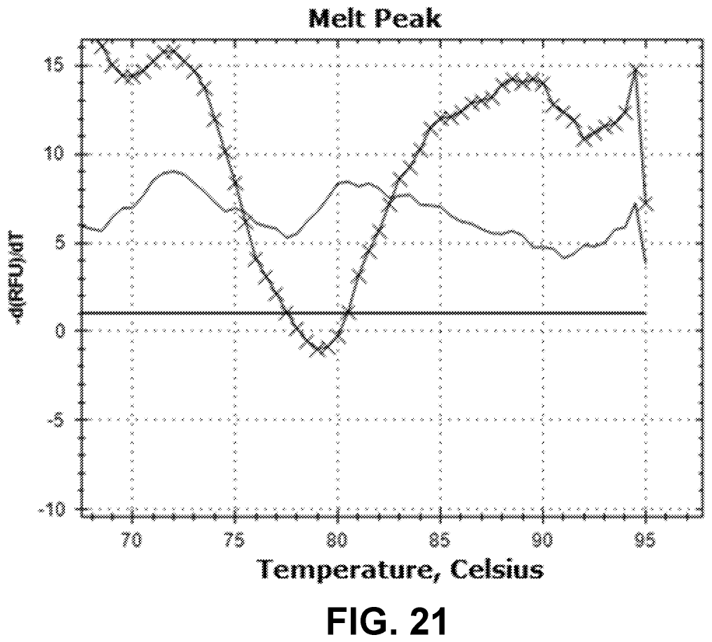

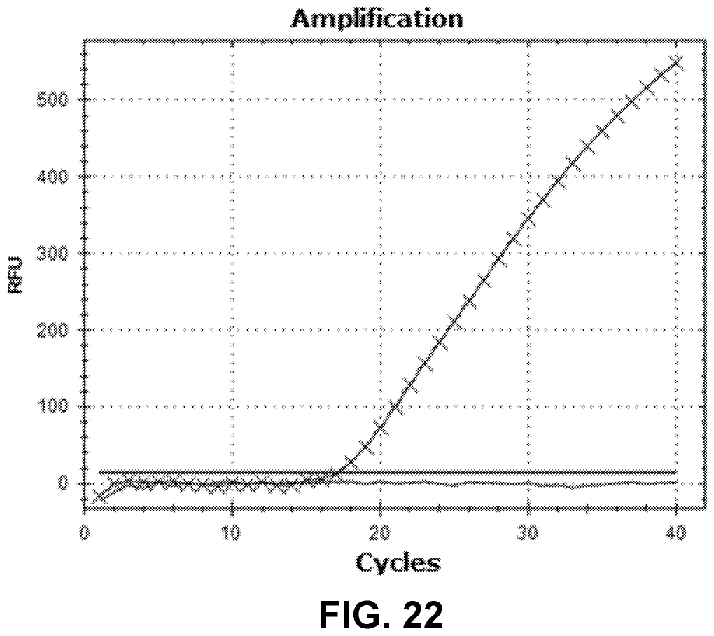

The following example shows results of PCR reactions comprising 2 different designs of PTO (tagging probes) specific for the ipaH gene, carrying a loop-design as the MTDR region where the last part of the PTO (tagging probe) comprises the targeting portion (quenching probe part), separated from the MTDR region by a loop region. All reactions are performed with the 2 common primers (DEC229F and DEC230R). The 2 different PTOs (tagging probes) were tested alone (RMD7P, FIGS. 19-20 and RMD8P, FIGS. 21-22). The results illustrate that each of the probes performs well in PCR and provide amplification curves in the presence of the specific target only. In addition, the results show that both probes provide a melting curve in the presence of the correct target but no target in the NTC.

TABLE 3
Primer and probes sequences (5′ to 3′):

| | | Seq |
|---|---|---|
| | | ID |
| | | NO: |
| PCR | | |
| Primers: | | |
| DEC229F | ZGTCCATCAGGCATCAGAAGG | 1 |
| DEC230R | ZGGTAGACTTCTATCTCATCCAC | 2 |
| Quenching | | |
| probe | | |
| DEC481rP | GATACTAGAGTTTCATAGTTCGTAGTCAAGAT | 3 |
| | GATAGATTGGAAGTGCG-(dT-BHQ1)-CAG- | |
| | (dT-BHQ2)-CAG-BHQ3 | |
| Tagging | | |
| Probe | | |
| RMD7P | BHQ1-AATGTTCCGCC(dT-FAM)CGAAATTC | 13 |
| | TGGAGATATCGAACGCGAAAAAAAAAAAAAAC | |
| | GCGTTCG-phos | |
| RMD8P | BHQ1-AATGTTCCGCC(dT-FAM)CGAAATTC | 14 |
| | TGGAGATATCGAACGCGAAAACGCGT | |
| | (dT-BHQ1)CG-phos | |

Z = TINA, BHQ1 = black hole quencher 1, BHQ2 = black hole quencher 2, BHQ3 = black hole quencher 3, (dT-FAM) = internal FAM attached to T. (dT-BHQ1) = internal BHQ1 attached to T. (dT-BHQ2) = internal BHQ2 attached to T. phos = phosphate group to block extension.

### PCR Reaction:

A 10 ul reaction was prepared that contained 1×SsoAdvanced Universal Probes Supermix (prod. #172-5280, Bio-Rad), 200 nM forward and reverse primer, 400 nM tagging probe, 1:200 dilution of target, 0.25 U Uracil DNA Glycosylase, 1 U/μL, (#EN0361 Fermentas). Target was prepared by mixing a single colony of E. coli in 200 ul water and boiling 95° C. for 15 minutes. 25 ul boiled target was subsequently added to 100 ul sterile water as a target stock solution which was diluted 5× in the final PCR reaction. Reactions were assembled in AB gene SuperPlate 96-well PCR plate (kat.nr. AB2800) and sealed with Optically clear, adhesive, Microseal B film (Bio-Rad, Cat.nr. MSB 1001).

The PCR reaction mix was subjected to the following PCR cycling and melting curve program (Bio-Rad CFX96 Real-Time PCR Detection System):

TABLE 4
PCR Program

| | Temp (° C.) | Time | |
|---|---|---|---|
| 1 | 40 | for 10 minutes | UNG treatment |
| 2 | 95 | for 10 minutes | Activation/denaturation |
| 3 | 95 | for 15 seconds | a) Denaturation |

Plate Read

| 4 | 60 | for 60 seconds | b) Annealing/elongation |
|---|---|---|---|
| | GoTo 3, 39 more times | | |
| 5 | 95 | for 10 seconds | |
| 6 | Melt Curve 40° C., to 95° C., increment 0.5° C., for 5 seconds | | |
| 7 | 10 | for 10 minutes | |

End

### Example 4: Testing a Different MTDR

In this experiment we tested some PTO probes having a different sequence than in the previous examples (RMD) and a matching quencher probe (CQO) to elucidate if a different sequence might work less efficiently to bind the polymerase. The primers and the probe target were the same as for the IpaH probes. As was done for the IpaH probe measurements, a normal hydrolysis probe assay was tested, mValidPrime, with RMD PTO probes and quenchers also present in the samples. With this test we want to evaluate if also the qPCR reaction of this assay becomes inhibited due to limited access of polymerase.

### Methods

qPCR

The experiment was performed on a LightCycler 480 instrument (Roche). gBlocks, used as template for the RMD assay (Table 5) and for the mValidPrime assay, were ordered from IDT. The master mix was TATAA Probe GrandMaster mix. Taq DNA Polymerase was added to the master mix to produce polymerase concentrations 1, 2.5, 5, and 10 times that of normal polymerase concentration in the master mix, where a normal concentration is defined as the concentration indicated by the manufacturer as the optimal concentration for performing the reaction. The qPCR measurement was performed with five probes for the RIMD assay (Table 6). Each probe was tested in ten different mixtures, as shown in Table 7. One set of five mixtures, with four different concentrations of polymerase and one NTC, was made with only the RMD system. One set of five other mixtures was made without IpaH primers and template, but with presence of primers, probes and template of the assay ValidPrime for mouse. The reagents were mixed to the concentrations shown in Table 8. All samples were run in duplicates. The temperature program is shown in Table 9. Here the fluorescence was measured both in step 2 (60° C.) and in step 3 (95° C.) of the program. The fluorescence is normally only measured in the 60° C. step. Since the probes might be hybridized to the quenching (CQO) probes and the fluorescence might be quenched at 60° C., we here also measured at 95° C. where all double stranded DNA is melted.

TABLE 5
Sequences of gBlocks.

RMD sequence (SEQ ID NO: 15):
GAGGACCGTGTCGCGCTCACATGGAACAATCTCCGGAAAACCCTCCTG
GTCCATCAGGCATCAGAAGGCCTTTTCGATAATGATACCGGCGCTCTG
CTCTCCCTGGGCAGGGAAATGTTCCGCCTCGAAATTCTGGAGGACATT
GCCCGGGATAAAGTCAGAACTCTCCATTTTGTGGATGAGATAGAAGTC
TACCTGGCCTTCCAGACCATGCTCGCAGAGAAACTTCAGCTCTCTACT
GCCGTGAAGGAAATGCGTTTCTATGGCGTGTCGGGAGTGACAGCAAAT
GACCTCCGCACTGCCGAAGCCATGGTCAGAAGCCGTGAAGAGAATGAA
TTTACGGACTGGTTCTCCCTCTGG
Color code: Forward primer; Reverse primer; Probe target?

TABLE 6
Sequences of probes and primers

| | | | | SEQ |
|---|---|---|---|---|
| Type | Name | Length | Sequence | ID NO: |
| forward | DEC229F | 20 | GTCCATCAGGCATCAGAAGG | 1 |
| primer | | | | |
| reverse | DEC230R | 22 | GGTAGACTTCTATCTCATCCA | 2 |
| primer | | | C | |
| quencher | RMD23rP | 57 | GATACTAGAGTTTCATAGTTC | 16 |
| probe | | | GTAGTCAAGATGATAGATTGG | |
| (CQO) | | | AAGTGCG(dT-BHQ-1)CAG | |
| | | | (dT-BHQ-2)CAG-BHQ-3 | |
| Tagging | RMD28P | 46 | BHQ1-AATGTTCCGCC(dT-F | 17 |
| probe | | | AM)CGAAATTCTGGAGTATAC | |
| (PTO) | | | GGCCGATTAGA TATAG- | |
| | | | phos | |
| Tagging | RMD29P | 51 | BHQ1-AATGTTCCGCC(dT-F | 18 |
| probe | | | AM)CGAAATTCTGGAGTATA | |
| (PTO) | | | CGGCCGATTAGA | |
| | | | TATAGAATGG-phos | |
| Tagging | RMD30P | 58 | BHQ1-AATGTTCCGCC(dTF- | 19 |
| probe | | | AM)CGAAATTCTGGAGTATA | |
| (PTO) | | | CGGCCGATTAGA | |
| | | | TATAGAATGGATATCGC- | |
| | | | phos | |
| Tagging | RMD31P | 74 | BHQ1-AATGTTCCGCC(dT-F | 20 |
| probe | | | AM)CGAAATTCTGGAGTATAC | |
| (PTO) | | | GGCCGATTAGA | |
| | | | TATAGAATGGATATCGCTATA | |
| | | | GATCTTATTCGG-phos | |
| Tagging | RMD32P | 91 | BHQ1-AATGTTCCGCC(dT- | 21 |
| probe | | | FAM)CGAAATTCTGGAGTATA | |
| (PTO) | | | CGGCCGATTAGA | |
| | | | TATAGAATGGATATCGCTATA | |
| | | | GATCTTATTCGGTTAAGATAG | |
| | | | TTGTAGGC-phos | |

TABLE 7
Mixing protocol. Volumes are in μL.

| | | TATAA | | | | | | | | |
|---|---|---|---|---|---|---|---|---|---|---|
| Reac- | | Grand | | | | | mValid | mValid | mValid | |
| tion | | Master | Quencher | RMD | RMD | RMD | Prime | Prime | Prime | |
| Mix | H₂O | mix | probe | Probe | Primers | Template | Probe | Primers | Template | Polymerase |
| A | 1.8 | 5 | 0.8 | 2 | 0.2 | 0.2 | — | — | — | — |
| B | 1.7 | 5 | 0.8 | 2 | 0.2 | 0.2 | — | — | — | 0.06 |
| C | 1.6 | 5 | 0.8 | 2 | 0.2 | 0.2 | — | — | — | 0.16 |
| D | 1.4 | 5 | 0.8 | 2 | 0.2 | 0.2 | — | — | — | 0.36 |
| E | 2.0 | 5 | 0.8 | 2 | 0.2 | — | — | — | — | — |
| F | 1.6 | 5 | 0.8 | 2 | — | — | 0.2 | 0.2 | 0.2 | — |
| G | 1.5 | 5 | 0.8 | 2 | — | — | 0.2 | 0.2 | 0.2 | 0.06 |
| H | 1.4 | 5 | 0.8 | 2 | — | — | 0.2 | 0.2 | 0.2 | 0.16 |
| I | 1.2 | 5 | 0.8 | 2 | — | — | 0.2 | 0.2 | 0.2 | 0.36 |
| J | 1.8 | 5 | 0.8 | 2 | — | — | 0.2 | 0.2 | — | — |

TABLE 8
Reagent concentrations

| | | |
|---|---|---|
| | Working | |
| Reagents | solution conc. | Final conc. |

| | | | | |
|---|---|---|---|---|
| Primers (forward & | 10 | μM | 200 | nM |
| reverse) RMD | | | | |
| PTO Probe RMD | 2 | μM | 400 | nM |
| Quencher probe | 10 | μM | 800 | nM |
| Template (gBlocks) | 1*10⁶ | molecules/μl | 2*10⁴ | molecules/μl |
| RMD | | | | |
| Primers (forward & | 10 | μM | 200 | nM |
| reverse) | | | | |
| mValidPrime | | | | |
| Probe mValidPrime | 10 | μM | 200 | nM |
| Template (gBlocks) | 1*10⁶ | molecules/μl | 2*10⁴ | molecules/μl |
| mValidPrime | | | | |

| | | |
|---|---|---|
| Master Mix | 2X | 1X |

| | | | |
|---|---|---|---|
| Taq polymerase | 5 | units/μl | varying |

TABLE 9
Temperature program for the probe qPCR run.

| Step | Time | Temperature | Cycles |
|---|---|---|---|
| Activation | 60 s | 95° C. | 50 |
| Cycling | 15 s | 95° C. (Data acquisition) | |
| Denaturation | 60 s | 60° C. (Data acquisition) | |
| Extension | | | |
| Melt curve | 0.5° C./10 s | 40° C. − 95° C. (Data acquisition) | |
| Cooling | 600 s | 10° C. | |

### Results

### CQ-Values

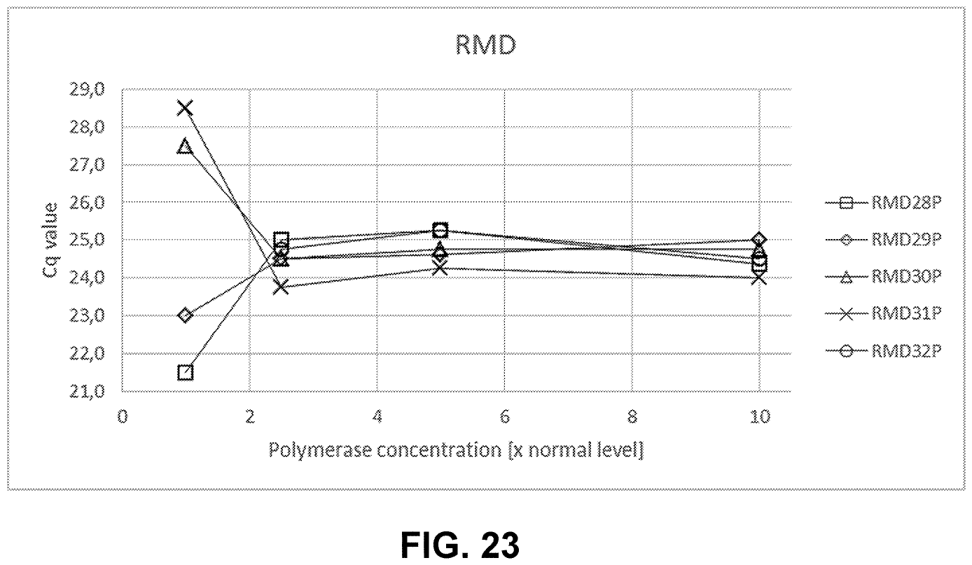

The replicate averages of the Cq values for the RMD samples (A-D) were calculated. They are plotted as a function of polymerase concentration in FIG. 23. Amplification generally increased with increasing concentrations of polymerase.

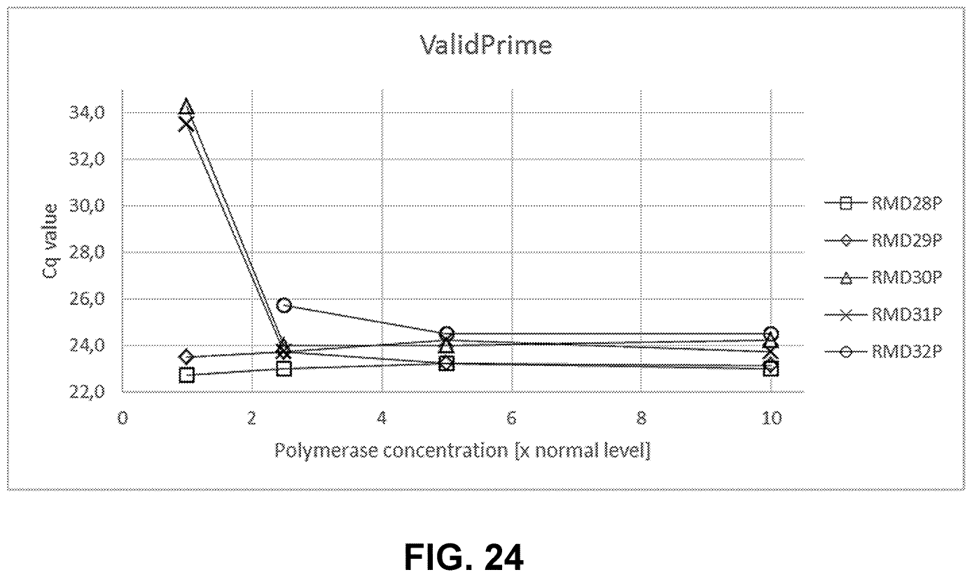

FIG. 24 shows the Cq values of the samples containing the mValidPrime probes. The samples contain also the various RMD probes, but since no primers or templates for these probes were in the samples, only the mValidPrime target is amplified and the fluorescence mainly comes from the mValidPrime probes. Amplification generally increased with increasing concentrations of polymerase.

### Amplification Curves

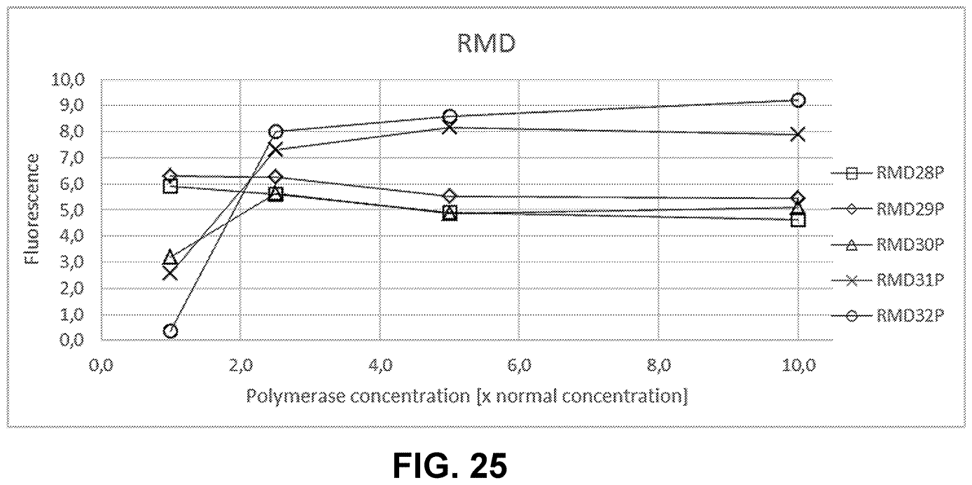

The amplitude of the amplification curves can be adjusted by varying the length of the probes and the polymerase concentration, as shown in FIG. 25. This figure shows the fluorescence amplitudes measured in the last amplification cycle in the denaturation step at 95° C. Generally, at higher polymerase concentrations higher amplitudes were reached for all probes.

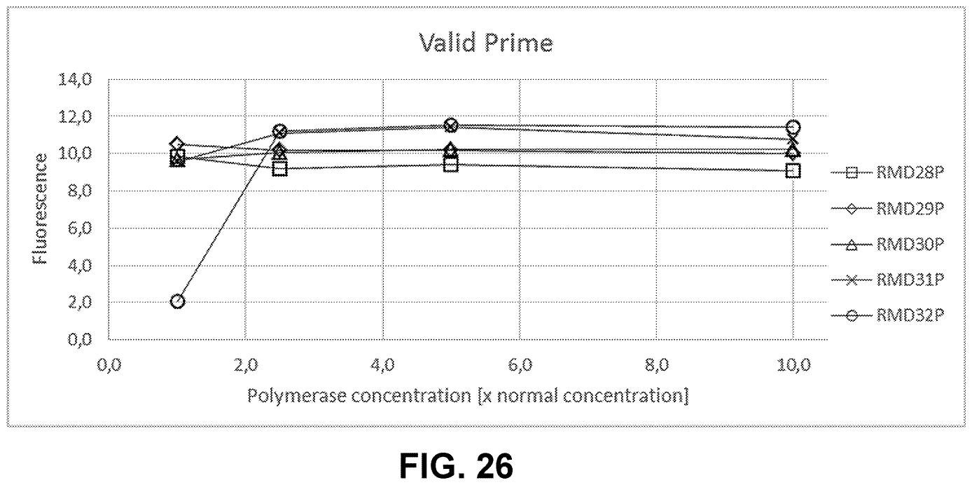

The amplitude of the mValidPrime probes, FIG. 26, were essentially stable over the tested range of polymerase concentrations.

### Melting Temperatures

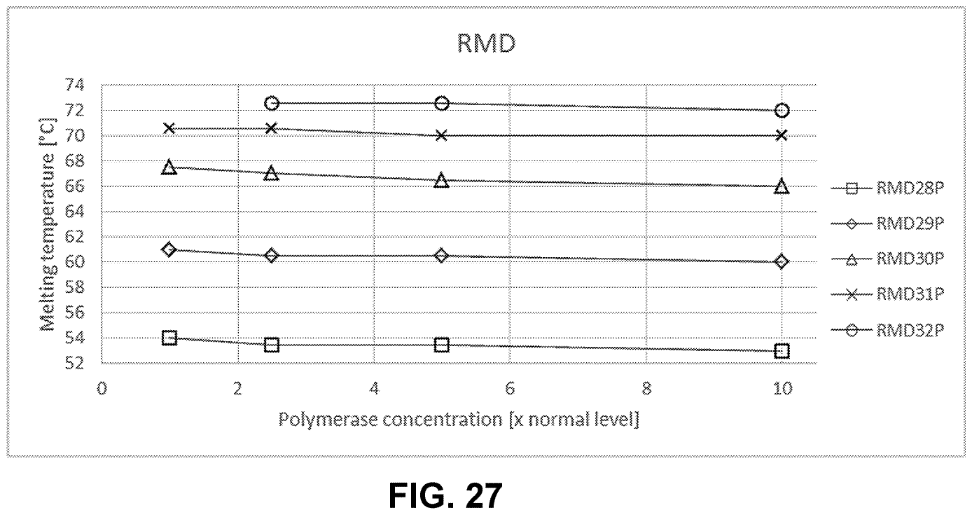

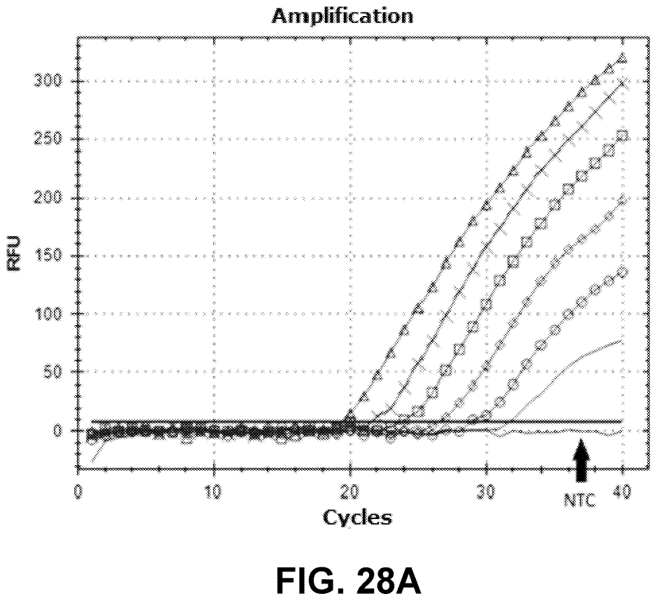

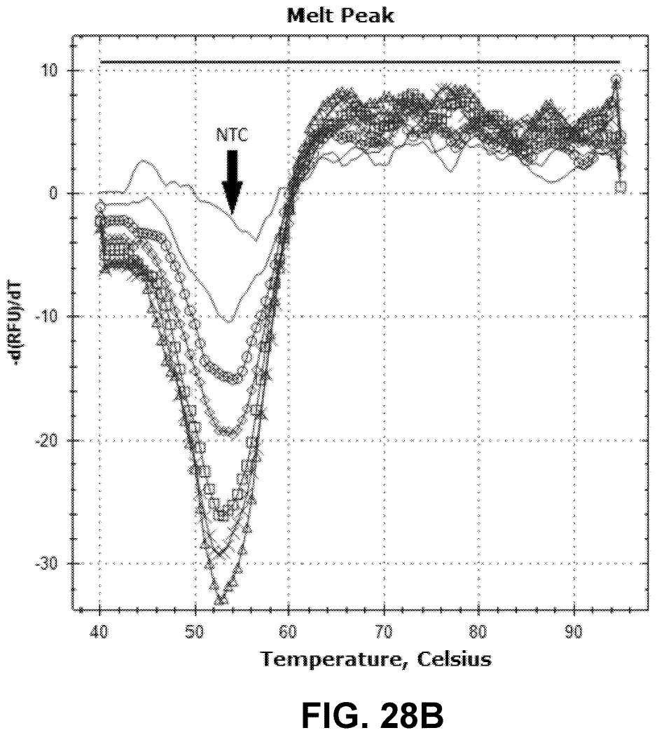

The measured melting temperatures of the RMD probes are found in FIG. 27. Melting curves were only observed for the samples containing RMD template, mixtures A, B, C and D. The melting temperatures increased with probe length, and they decreased slightly with increasing polymerase concentration.

### Example 5: Target Concentration

The following example shows results of PCR reactions comprising 5× dilution curves of 6 different target concentrations detected by the Probing and Tagging oligonucleotide (PTO) DEC486P specific for the ipaH gene. Reactions were performed with the 2 common primers (DEC229F and DEC230R). The Probing and Tagging oligonucleotides were tested in combination with a single Capture and Quenching probe design (DEC481rP). The results illustrate that the Probing and Tagging probe works by providing an amplification curve and a melting curve corresponding to the target dilution. As can be seen from the NTC reactions the DEC486P probe showed very little background.

TABLE 10
Primer and probes sequences (5′ to 3′):

| | | Seq ID |
|---|---|---|
| | | NO: |
| PCR | | |
| Primers: | | |
| DEC229F | ZGTCCATCAGGCATCAGAAGG | 1 |
| DEC230R | ZGGTAGACTTCTATCTCATCCAC | 2 |
| Capturing | | |
| and | | |
| Quenching | | |
| probe | | |
| DEC481rP | GATACTAGAGTTTCATAGTTCGTAGTCAAGAT | 3 |
| | GATAGATTGGAAGTGCG-(dT-BHQ1)-CAG- | |
| | (dT-BHQ2)-CAG-BHQ3 | |
| Probing | | |
| and | | |
| Tagging | | |
| Probe | | |
| DEC486P | BHQ1-AATGTTCCGCC(dT-FAM) | |
| | CGAAATTCTGGAGTATATCGACTGACGCACTT | |
| | CCAA-phos | 4 |

Z = TINA, BHQ1 = black hole quencher 1, BHQ2 = black hole quencher 2, BHQ3 = black hole quencher 3, (dT-FAM) = internal fluorescein label attached to T. FAM = Fluorescein. (dT-BHQ1) = internal BHQ1 attached to T. (dT-BHQ2) = internal BHQ2 attached to T. phos = phosphate group to block extension.

PCR reaction: A 10 pl reaction was prepared that contained 1×Sso Advanced Universal Probe Supermix (prod. #Promega A6101), 200 nM forward and reverse primer, 400 nM Probing and Tagging probe, 800 nM Capturing and Quenching probe, 1:200 dilution of target, 0.25 U Uracil DNA Glycosylase, 1 U/μL, (#EN0361 Fermentas). Target was prepared by mixing a single colony of E. coli carrying the ipaH gene in 200 pl water and boiling 95° C. for 15 minutes. 25 pl boiled target was subsequently added to 100 pl sterile water as a target stock solution which was diluted 5 to 15625× in the final PCR reaction. Reactions were assembled in AB gene SuperPlate 96-well PCR plate (kat.nr. AB2800) and sealed with Optically clear, adhesive, Microseal B film (Bio-Rad, Cat.nr. MSB1001).

PCR reaction mix was subjected to the following PCR cycling and melting curve program (Bio-Rad CFX96 Real-Time PCR Detection System):

TABLE 11
PCR program:

| | Temp (° C.) | Time | |
|---|---|---|---|
| 1 | 40 | for 10 minutes | UNG treatment |
| 2 | 95 | for 10 minutes | Activation/denaturation |
| 3 | 95 | for 15 seconds | a) Denaturation |

Plate Read

| 4 | 60 | for 60 seconds | b) Annealing/elongation |
|---|---|---|---|
| | GoTo 3, 39 more times | | |
| 5 | 95 | for 10 seconds | |
| 6 | Melt Curve 40° C., to 95° C., increment 0.5° C., for 5 seconds | | |
| 7 | 10 | for 10 minutes | |

End

### REFERENCES

Kibbe W A. ‘OligoCalc: an online oligonucleotide properties calculator’. (2007) Nucleic Acids Res. 35(webserver issue): May 25.
BioTechniques 38:569-575 (April 2005)
Nucleic Acids Symp Ser (2008) 52(1): 47-48.

It is claimed:

1. A method for detecting a nucleic acid sequence, comprising:
(a) hybridizing a nucleic acid sequence with a probe comprising (i) a targeting portion comprising a nucleotide sequence substantially complementary to the nucleic acid sequence, and (ii) a melting temperature deciding region (MTDR) comprising a nucleotide sequence non-complementary to the nucleic acid sequence, and (iii) at least one set of interactive labels comprising at least one fluorophore and at least one quencher;
(b) hybridizing said probe with a quenching probe comprising (i) a capturing portion comprising a nucleotide sequence which is reverse complementary to the MTDR of the probe and (ii) at least one quenching molecule, wherein the MTDR of the probe is configured to hybridize with the capturing portion of the quenching probe to form a tag duplex;
(c) contacting the tag duplex with an enzyme having nuclease activity to induce cleavage of the tag duplex and release of an activated tag duplex fragment comprising the MTDR hybridized to the capturing portion and the at least one fluorophore;
(d) melting or hybridizing said activated tag duplex fragment to obtain a signal from the at least one fluorophore, and
(e) detecting the signal to determine presence of the nucleic acid sequence.

2. The method of claim 1, wherein (b) is performed prior to (a) and the hybridizing of (a) comprises hybridizing the nucleic acid with the targeting portion of the probe in the tag duplex.

3. The method of claim 1, wherein the capturing portion of the quenching probe hybridizes with a second MTDR of second probe, where the second MTDR is different from the MTDR of the probe in (a).

4. The method of claim 1, wherein said method is conducted in the presence of a primer pair, said primer pair comprising a first primer complementary to the nucleic acid and which primes synthesis of a first extension product that is complementary to the nucleic acid, and a second primer complementary to said first extension product and which primes synthesis of a second extension product.

5. The method of claim 1, wherein the targeting portion and the MTDR of the probe are separated by a linker molecule.

6. The method of claim 5, wherein the linker is a nucleic acid linker comprising between about 1-200 nucleotides or an organic compound.

7. The method of claim 1, wherein the probe comprises between about 10 and 500 nucleotides.

8. The method of claim 1, wherein quenching probe comprises between about 10 and 500 nucleotides.

9. The method of claim 1, wherein the probe has a 5′ end and the targeting portion is located in the 5′ end of the probe.

10. The method of claim 1, wherein the probe has a 3′ end and the MTDR is located in the 3′ end of the probe.

11. The method of claim 1, wherein the probe has a 3′ end, and the probe further comprises a blocking group in the 3′ end.

12. The method of claim 11, wherein the blocking group is selected from the group consisting of biotin, labels, a phosphate group, alkyl group, non-nucleotide linker, phosphorothioate, and/or alkane-diol and/or wherein the blocking group comprises nucleotide with no 3′-hydroxyl group such as dideoxynucleotide.

13. The method of claim 1, wherein the quenching probe has a 3′ end, and the quenching probe further comprises a blocking group in the 3′ end.

14. The method of claim 13, wherein the blocking group is selected from the group consisting of biotin, labels, a phosphate group, alkyl group, non-nucleotide linker, phosphorothioate, and/or alkane-diol and/or wherein the blocking group comprises nucleotide with no 3′-hydroxyl group such as dideoxynucleotide.

15. The method of claim 1, wherein the enzyme having nuclease activity is a template dependent DNA polymerase.

16. The method of claim 15, wherein the DNA polymerase is a thermostable template dependent DNA polymerase.

17. A kit of parts for detecting at least one nucleic acid sequence from a nucleic acid mixture, the kit comprising:
i. at least one probe comprising (i) a targeting portion comprising a nucleotide sequence substantially complementary to a nucleic acid sequence in the nucleic acid mixture, and (ii) a melting temperature deciding region (MTDR) comprising a nucleotide sequence non-complementary to the nucleic acid sequence, and (iii) at least one set of interactive labels comprising a fluorophore and a quencher, and
ii. at least one quenching probe comprising (i) a capturing portion comprising a nucleotide sequence which is reverse complementary to the MTDR and (ii) a quencher, and
iii. optionally instructions for detecting the nucleic acid sequence.

18. A reaction mixture, comprising:
at least one pair of primers, at least one probe and at least one quenching probe,
wherein said pair of primers comprises a first a primer complementary to a nucleic acid and which primes synthesis of a first extension product that is complementary to the nucleic acid, and a second primer complementary to the first extension product and which primes synthesis of a second extension product;
wherein said at least one probe comprises (i) a targeting portion comprising a nucleotide sequence substantially complementary to the nucleic acid or to the complement of the nucleic acid, and (ii) a melting temperature deciding region (MTDR) comprising a nucleotide sequence non-complementary to the nucleic acid, and (iii) at least one set of interactive labels comprising a fluorophore and a quencher; and
wherein said at least one quenching probe comprises (i) a capturing portion comprising a nucleotide sequence which is reverse complementary to the MTDR and (ii) a quencher;
wherein the targeting portion hybridizes to the nucleic acid in a region between one member of said primer pair and the complement of the other member of said primer pair.

19. A method for detecting a nucleic acid sequence, comprising:
(a) hybridizing a nucleic acid sequence with a probe comprising (i) a targeting portion comprising a nucleotide sequence substantially complementary to the nucleic acid sequence, and (ii) a melting temperature deciding region (MTDR) comprising a nucleotide sequence non-complementary to the nucleic acid sequence, and (iii) at least one set of interactive labels comprising a fluorophore and a quencher;
(b) contacting the hybridized nucleic acid sequence-probe with an enzyme having nuclease activity to induce cleavage of a fragment comprising the MTDR and the at least one fluorophore;
(c) hybridizing the fragment with a quenching probe to form an activated tag duplex, the quenching probe comprising (i) a capturing portion comprising a nucleotide sequence which is reverse complementary to the MTDR and (ii) a quencher;
(d) melting or hybridizing the activated tag duplex to obtain a signal from the fluorophore, and
(e) detecting the signal to determine presence of the nucleic acid sequence.


SEQUENCE LISTING (21 sequences)

SEQ ID NO: 1 - DNA, 20 bp, synthetic construct
  misc_feature 1..20: note = Primer
  misc_feature 1: note = TINA (twisted intercalating nucleic acid)
  source 1..20: mol_type = other DNA
gtccatcaggcatcagaagg

SEQ ID NO: 2 - DNA, 22 bp, synthetic construct
  misc_feature 1..22: note = Primer
  misc_feature 1: note = TINA (twisted intercalating nucleic acid)
  source 1..22: mol_type = other DNA
ggtagacttctatctcatccac

SEQ ID NO: 3 - DNA, 57 bp, synthetic construct
  misc_feature 1..57: note = Quenching probe
  misc_feature 50: note = BHQ1= black hole quencher 1
  misc_feature 54: note = BHQ2= black hole quencher 2
  misc_feature 57: note = BHQ3= black hole quencher 3
  source 1..57: mol_type = other DNA
gatactagagtttcatagttcgtagtcaagatgatagattggaagtgcgtcagtcag

SEQ ID NO: 4 - DNA, 48 bp, synthetic construct
  misc_feature 1..48: note = Tagging probe
  misc_feature 1: note = BHQ1= black hole quencher 1
  misc_feature 12: note = FAM
  misc_feature 48: note = phosphate
  source 1..48: mol_type = other DNA
aatgttccgcctcgaaattctggagtatatcgactgacgcacttccaa

SEQ ID NO: 5 - DNA, 48 bp, synthetic construct; identical to SEQ ID NO: 4
  misc_feature 1..48: note = Tagging probe
  misc_feature 6: note = BHQ1= black hole quencher 1
  misc_feature 18: note = FAM
  misc_feature 48: note = phosphate
  source 1..48: mol_type = other DNA

SEQ ID NO: 6 - DNA, 48 bp, synthetic construct; identical to SEQ ID NO: 4
  misc_feature 1..48: note = Tagging probe
  misc_feature 1: note = BHQ1= black hole quencher 1
  misc_feature 18: note = FAM
  misc_feature 48: note = phosphate
  source 1..48: mol_type = other DNA

SEQ ID NO: 7 - DNA, 48 bp, synthetic construct; identical to SEQ ID NO: 4
  misc_feature 1..48: note = Tagging probe
  misc_feature 1: note = BHQ1= black hole quencher 1
  misc_feature 21: note = FAM
  misc_feature 48: note = phosphate
  source 1..48: mol_type = other DNA

SEQ ID NO: 8 - DNA, 48 bp, synthetic construct; identical to SEQ ID NO: 4
  misc_feature 1..48: note = Tagging probe
  misc_feature 1: note = BHQ1= black hole quencher 1
  misc_feature 26: note = FAM
  misc_feature 48: note = phosphate
  source 1..48: mol_type = other DNA

SEQ ID NO: 9 - DNA, 25 bp, synthetic construct
  misc_feature 1..25: note = TaqMan probe
  misc_feature 1: note = FAM
  misc_feature 25: note = BHQ1= black hole quencher 1
  source 1..25: mol_type = other DNA
aatgttccgcctcgaaattctggag

SEQ ID NO: 10 - DNA, 48 bp, synthetic construct
  misc_feature 1..48: note = Tagging probe
  misc_feature 1: note = BHQ1= black hole quencher 1
  misc_feature 12: note = FAM
  misc_feature 48: note = phosphate
  source 1..48: mol_type = other DNA
aatgttccgcctcgaaattctggagtatactgactgacgcacttccaa

SEQ ID NO: 11 - DNA, 57 bp, synthetic construct
  misc_feature 1..57: note = Tagging probe
  misc_feature 1: note = BHQ1= black hole quencher 1
  misc_feature 12: note = FAM
  misc_feature 57: note = phosphate
  source 1..57: mol_type = other DNA
aatgttccgcctcgaaattctggagtatactgactgacgcacttccaatctatcatc

SEQ ID NO: 12 - DNA, 66 bp, synthetic construct
  misc_feature 1..66: note = Tagging probe
  misc_feature 1: note = BHQ1= black hole quencher 1
  misc_feature 12: note = FAM
  misc_feature 66: note = phosphate
  source 1..66: mol_type = other DNA
aatgttccgcctcgaaattctggagtatactgactgacgcacttccaatctatcatcttgactacg

SEQ ID NO: 13 - DNA, 59 bp, synthetic construct
  misc_feature 1..59: note = Tagging sequence
  misc_feature 1: note = BHQ1= black hole quencher 1
  misc_feature 12: note = FAM
  misc_feature 59: note = phosphate
  source 1..59: mol_type = other DNA
aatgttccgcctcgaaattctggagatatcgaacgcgaaaaaaaaaaaaaacgcgttcg

SEQ ID NO: 14 - DNA, 49 bp, synthetic construct
  misc_feature 1..49: note = Tagging probe
  misc_feature 1: note = BHQ1= black hole quencher 1
  misc_feature 12: note = FAM
  misc_feature 47: note = BHQ1= black hole quencher 1
  misc_feature 49: note = phosphate
  source 1..49: mol_type = other DNA
aatgttccgcctcgaaattctggagatatcgaacgcgaaaacgcgttcg

SEQ ID NO: 15 - DNA, 360 bp, synthetic construct
  misc_feature 1..360: note = RMD sequence
  misc_feature 1..360: note = RMD sequence
  source 1..360: mol_type = other DNA
gaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctctactgccgtgaaggaaatgcgtttctatggcgtgtcgggagtgacagcaaatgacctccgcactgccgaagccatggtcagaagccgtgaagagaatgaatttacggactggttctccctctgg

SEQ ID NO: 16 - DNA, 57 bp, synthetic construct; identical to SEQ ID NO: 3
  misc_feature 1..57: note = Quencher probe
  misc_feature 50: note = BHQ1= black hole quencher 1
  misc_feature 54: note = BHQ2= black hole quencher 2
  misc_feature 57: note = BHQ3= black hole quencher 3
  source 1..57: mol_type = other DNA

SEQ ID NO: 17 - DNA, 46 bp, synthetic construct
  misc_feature 1..46: note = Probe
  misc_feature 1: note = BHQ1= black hole quencher 1
  misc_feature 12: note = FAM
  misc_feature 46: note = phosphate
  source 1..46: mol_type = other DNA
aatgttccgcctcgaaattctggagtatacggccgattagatatag

SEQ ID NO: 18 - DNA, 51 bp, synthetic construct
  misc_feature 1..51: note = Probe
  misc_feature 1: note = BHQ1= black hole quencher 1
  misc_feature 12: note = FAM
  misc_feature 51: note = phosphate
  source 1..51: mol_type = other DNA
aatgttccgcctcgaaattctggagtatacggccgattagatatagaatgg

SEQ ID NO: 19 - DNA, 58 bp, synthetic construct
  misc_feature 1..58: note = Probe
  misc_feature 1: note = BHQ1= black hole quencher 1
  misc_feature 12: note = FAM
  misc_feature 58: note = phosphate
  source 1..58: mol_type = other DNA
aatgttccgcctcgaaattctggagtatacggccgattagatatagaatggatatcgc

SEQ ID NO: 20 - DNA, 74 bp, synthetic construct
  misc_feature 1..74: note = Probe
  misc_feature 1: note = BHQ1= black hole quencher 1
  misc_feature 12: note = FAM
  misc_feature 74: note = phosphate
  source 1..74: mol_type = other DNA
aatgttccgcctcgaaattctggagtatacggccgattagatatagaatggatatcgctatagatcttattcgg

SEQ ID NO: 21 - DNA, 91 bp, synthetic construct
  misc_feature 1..91: note = Probe
  misc_feature 1: note = BHQ1= black hole quencher 1
  misc_feature 12: note = FAM
  misc_feature 91: note = phosphate
  source 1..91: mol_type = other DNA
aatgttccgcctcgaaattctggagtatacggccgattagatatagaatggatatcgctatagatcttattcggttaagatagttgtaggc